(19) United States
(12) Patent Application Publication
(10) Pub. No.: US 2016/0143772 A1
(43) Pub. Date: May 26, 2016

(54) THERMAL THERAPEUTIC GARMENTS

(71) Applicant: Cynthia MCLAIN, Denver, CO (US)

(72) Inventor: Cynthia MCLAIN, Denver, CO (US)

(21) Appl. No.: 14/946,539

(22) Filed: Nov. 19, 2015

Related U.S. Application Data
(60) Provisional application No. 62/082,265, filed on Nov. 20, 2014.

(51) Int. Cl. A61F 7/02; A61F 5/01

(52) U.S. Cl. CPC A61F 7/02; A61F 5/0118; A61F 2007/0037

(57) ABSTRACT

A therapeutic digit cover according to an embodiment of the present invention includes a tubular sleeve for one or more digits, wherein the tubular sleeve has an opening and a length and is closed along at least part of the length; a thermal pack incorporated in the tubular sleeve, the thermal pack is fully enclosed in the tubular sleeve such that there is no direct contact of the thermal pack with a user's skin when the therapeutic digit cover is worn by the user; a strap with a closed loop, wherein the closed loop is configured to encircle a palm of the hand between an index finger and a thumb of the user when the therapeutic digit cover is worn by the user; and at least one guide loop attachment to hold the strap in place on the user's hand.

### CROSS-REFERENCE TO RELATED APPLICATIONS

This application claims the benefit of U.S. Provisional Patent Application Ser. No. 62/082,265, filed on Nov. 20, 2014, which is incorporated by reference herein in its entirety for all purposes.

### TECHNICAL FIELD

The present invention relates generally to a therapeutic garment. More specifically, the invention relates to a therapeutic garment that can be heated or cooled to help relieve the symptoms of various injuries in different body members, and methods of treatment.

### BACKGROUND

Therapeutic garments can be used to relieve the symptoms of injuries to various body members such as hands, fingers, thumbs, elbow, feet, knees, ankles, low back, or shoulders. Examples of such injuries include Carpal Tunnel Syndrome, fatigue, Cumulative Trauma Syndrome, Repetitive Motion Syndrome, Plantar Fasciitis, Morton's neuroma, bone spurs, tendonitis, sprains, arthritis, and the like. Further examples of such injuries include injuries requiring treatment due to recreational and professional sports, as well as jobs requiring manual labor. These injuries can be reoccurring as a result of repetitive and continuous use of computers and electronic devices. To treat these symptoms, therapeutic garments can be either heated or cooled and applied directly to the injured site.

Typically, therapeutic garments have the ability to target the acute problem site of the injury. Beyond the “acute problem site,” damage to nearby muscles, nerves, and soft tissue continues to occur. It is preferable that the therapeutic garment treat both the “acute problem site” as well as its surrounding area.

Another challenge to existing therapeutic garments is that they typically use hook and loop fasteners to secure the therapeutic wrap to the body. For a person with diminished finger strength or limited manual dexterity, the hook and loop fastener can be hard to negotiate. The hook and loop fastener can snag onto nearby fabric, making it hard to position the therapeutic garment to the body member. Once the hook and loop fastener is attached in one location, the person must completely disengage it and re-fasten it to make any adjustments. Furthermore, over time, the hook and loop fastener can wear out.

### SUMMARY

A therapeutic digit cover according to an embodiment of the present invention includes a tubular sleeve for one or more digits, wherein the tubular sleeve has an opening and a length and is closed along at least part of the length; a thermal pack incorporated in the tubular sleeve, the thermal pack is fully enclosed in the tubular sleeve such that there is no direct contact of the thermal pack with a user's skin when the therapeutic digit cover is worn by the user; a strap with a closed loop, wherein the closed loop is configured to encircle a palm of the hand between an index finger and a thumb of the user when the therapeutic digit cover is worn by the user; and at least one guide loop attachment to hold the strap in place on the user's hand.

According to some embodiments, the tubular sleeve is conical. The tubular sleeve may have a pocket configured to accept the thermal pack, which may be, for example, an open pocket, a stitched closed pocket, and/or a pocket of a different color than that of the therapeutic digit cover, according to embodiments of the present invention. In some embodiments, the thermal pack comprises one or more of: gel, flax seed, rice, and corn. In some cases, the opening is a first opening, and the tubular sleeve has a second opening for a second digit of the hand of the user. According to some embodiments, the tubular sleeve is configured to accept at least one finger of the hand of the user. The tubular sleeve comprises elastic inserts.

According to some embodiments of the present invention, the guide loop attachment is a patch that allows for compression to be administered in specific areas of the user's hand. The guide loop attachment may vary in width, for example from 0.1 inches to 2 inches, according to embodiments of the present invention. The strap may be adjustable, and may, for example, include a ladder lock buckle for adjustment of the strap. In some cases, the tubular sleeve extends up to a distal phalanx of the digit.

A method of administering therapy to a digit of a user according to an embodiment of the present invention includes positioning a therapeutic digit cover comprising a tubular sleeve to cover at least one digit, wherein the tubular sleeve has an opening and is closed along at least part of its length; chilling or heating a thermal pack; securing the therapeutic digit cover with a strap, wherein the strap is a closed loop that encircles a palm of a hand between an index finger and a thumb of the user; adjusting the strap to apply pressure to a desired portion of the therapeutic digit cover.

In some cases, adjusting the strap includes tightening and loosening the strap using a ladder lock buckle fastener. Adjusting the strap includes looping the strap through one or more guide loop attachments to hold the strap in place, according to embodiments of the present invention. In some cases, the therapeutic digit cover is a first therapeutic digit cover, the method further comprising positioning a second therapeutic digit cover on the hand of the user.

A therapeutic digit cover kit according to an embodiment of the present invention includes: a first digit cover having a distal end and a proximal end, the proximal end comprising a first guide loop attachment; a second digit cover having a distal end and a proximal end, the proximal end comprising a second guide loop attachment, wherein the first digit cover is independent of the second digit cover; a strap configured to form a closed loop, wherein the closed loop is configured to encircle the palm of a hand of a user between an index finger and thumb of the user; wherein the strap is selectively insertable through one or both of the first guide loop and second guide loop attachment for customizing a number of digits covered. The strap may include a ladder lock buckle fastener.

According to some embodiments, the therapeutic digit cover kit further includes a third digit cover having a distal end and a proximal end, the proximal end comprising a third guide loop attachment, wherein the third digit cover is independent of the first and second digit covers, and wherein the strap is selectively insertable through one or more of the first, second, and third guide loop attachments for customizing the number of digits covered. According to some embodiment, the therapeutic digit cover kit further includes a fourth guide loop attachment, wherein the fourth digit cover is independent of the first, second, and third digit covers, and wherein the strap is selectively insertable through one or more of the first, second, third, and fourth guide loop attachments for customizing the number of digits covered.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 illustrates a posterior view of a single finger therapy garment, according to embodiments of the present invention.

FIG. 2 illustrates a posterior view of a single finger and thumb therapy garment, according to embodiments of the present invention.

FIG. 3 illustrates posterior view of a single finger and thumb therapy garment, according to embodiments of the present invention.

FIG. 4 illustrates an anterior view of the single finger and thumb therapy garment of FIG. 3, according to embodiments of the present invention.

FIG. 5 illustrates a posterior view of a hand mitt therapy garment (which may also be referred to as a “mini mitt”), according to embodiments of the present invention.

FIG. 6 illustrates an anterior view of the hand mitt therapy garment of FIG. 5 (which may also be referred to as a “mini mitt”), according to embodiments of the present invention.

FIG. 7 illustrates a posterior view of another hand mitt therapy garment, according to embodiments of the present invention.

FIG. 8 illustrates an anterior view of the hand mitt therapy garment of FIG. 7, according to embodiments of the present invention.

FIG. 9 illustrates a posterior view of the hand mitt therapy garment of FIGS. 7 and 8, according to embodiments of the present invention.

FIG. 10 illustrates an anterior view of the hand mitt therapy garment of FIGS. 7-9, according to embodiments of the present invention.

FIG. 11 illustrates a posterior view of the hand mitt therapy garment of FIGS. 7-10, according to embodiments of the present invention.

FIG. 12 illustrates a great toe therapy garment, according to embodiments of the present invention.

FIG. 13 illustrates a dorsal view of the great toe therapy garment of FIG. 12, according to embodiments of the present invention.

FIG. 14 illustrates a plantar view of the great toe therapy garment of FIGS. 12 and 13, according to embodiments of the present invention.

FIG. 15 illustrates a medial view of the great toe therapy garment of FIGS. 12-14, according to embodiments of the present invention.

FIG. 16 illustrates another medial view of the great toe therapy garment of FIGS. 12-15, according to embodiments of the present invention.

FIG. 17 illustrates a perspective view of a foot therapy garment, according to embodiments of the present invention.

FIG. 18 illustrates a dorsal view of the foot therapy garment of FIG. 17, according to embodiments of the present invention.

FIG. 19 illustrates a plantar view of the foot therapy garment of FIGS. 17 and 18, according to embodiments of the present invention.

FIG. 20 illustrates an elbow therapy garment, according to embodiments of the present invention.

FIG. 21 illustrates an anterior view of the elbow therapy garment of FIG. 20, according to embodiments of the present invention.

FIG. 22 illustrates a posterior view of the elbow therapy garment of FIGS. 20-21, according to embodiments of the present invention.

FIG. 23 illustrates a perspective view of a sock therapy garment, according to embodiments of the present invention.

FIG. 24 illustrates another perspective view of the sock therapy garment of FIG. 23, according to embodiments of the present invention.

FIG. 25 illustrates another perspective view of the sock therapy garment of FIGS. 23-24, according to embodiments of the present invention.

FIG. 26 illustrates another perspective view of the sock therapy garment of FIGS. 23-25, according to embodiments of the present invention.

FIG. 27 illustrates another perspective view of the sock therapy garment of FIGS. 23-26, according to embodiments of the present invention.

FIG. 28 illustrates another perspective view of the sock therapy garment of FIGS. 23-27, according to embodiments of the present invention.

FIG. 29 illustrates a bottom view of the sock therapy garment of FIGS. 23-28, according to embodiments of the present invention.

FIG. 30 illustrates a medial side view of the sock therapy garment of FIGS. 23-29, according to embodiments of the present invention.

FIG. 31 illustrates a perspective view of an ankle therapy garment, according to embodiments of the present invention.

FIG. 32 illustrates a dorsal view of the ankle therapy garment of FIG. 31, according to embodiments of the present invention.

FIG. 33 illustrates a plantar view of the ankle therapy garment of FIGS. 31-32, according to embodiments of the present invention.

FIG. 34 illustrates a back perspective view of the ankle therapy garment of FIGS. 31-33, according to embodiments of the present invention.

FIG. 35 illustrates a lateral side view of the ankle therapy garment of FIGS. 31-34, according to embodiments of the present invention.

FIG. 36 illustrates a medial side view of the ankle therapy garment of FIGS. 31-35, according to embodiments of the present invention.

FIG. 37 illustrates a knee therapy garment, according to embodiments of the present invention.

FIG. 38 illustrates an anterior view of the knee therapy garment of FIG. 37, according to embodiments of the present invention.

FIG. 39 illustrates a posterior view of the knee therapy garment of FIGS. 37-38, according to embodiments of the present invention.

FIG. 40 illustrates a posterior view of a lumbar/sacral therapy garment, according to embodiments of the present invention.

FIG. 41 illustrates an anterior view of the lumbar/sacral therapy garment of FIG. 40, according to embodiments of the present invention.

FIG. 42 illustrates a posterior view of the lumbar/sacral therapy garment of FIGS. 40-41, according to embodiments of the present invention.

FIG. 43 illustrates an anterior view of a shoulder therapy garment, according to embodiments of the present invention.

FIG. 44 illustrates an anterior view of the shoulder therapy garment of FIG. 43, according to embodiments of the present invention.

FIG. 45 illustrates a posterior view of the shoulder therapy garment of FIGS. 43-44, according to embodiments of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

Embodiments of the present invention include therapy garments which are more easily applied to the skin, are more comfortable once applied, are more conforming and/or customizable to a person's anatomy, and/or are more easily targeted to providing therapy to a specific portion of the person's anatomy. A therapy garment according to some embodiments of the present invention is easier to apply or adjust due to the use of adjustment mechanisms such as straps with ladder lock buckles. While other adjustment mechanisms are contemplated within the scope of the present invention, some people, particularly the elderly, the weak, or the infirm, may find that ladder lock buckle adjustment mechanisms are easier to operate and manipulate than the adjustment mechanisms included on such therapy garments currently on the market, according to embodiments of the present invention.

Existing therapy garment systems are often “one size fits all” and do not permit a high degree of customization. For example, existing therapy garments often include only one location that is intended to permit adjustment, rather than permitting the user to adjust the placement of a strap about the garment.

As used herein, reference numerals ending in the same two digits refer to similar structures throughout the various described embodiments, with a different hundreds or thousands digit prefix to signify that the structural element has or is capable of a function with respect to that embodiment that is similar to the function or capability described with respect to other embodiments. For example, elements ending in 00 refer to a therapy garment, elements ending in 10 refer to a tubular sleeve, elements ending in 15 refer to a first opening of a tubular sleeve, elements ending in 18 refer to an accommodation slit of a tubular sleeve, elements ending in 20 refer to a strap, elements ending in 30 refer to a guide loop attachment, elements ending in 40 refer to ladder lock buckles, elements ending in 50 refer to a pocket, elements ending in 60 refer to a thermal pack, elements ending in 70 refer to elastic inserts, elements ending in 80 refer to stitching, and elements ending in 90 refer to a flap, according to embodiments of the present invention. As such, any features and/or characteristics described with respect to one element used in an embodiment may apply to other such related or counterpart elements described with respect to other embodiments. The use of ladder lock buckles according to certain embodiments may permit the user to use some or all gel packs in order to customize the level of hot or cold therapy, or to customize the area over which the therapy is applied. Often, hook-and-loop closure systems do not permit such level of customization.

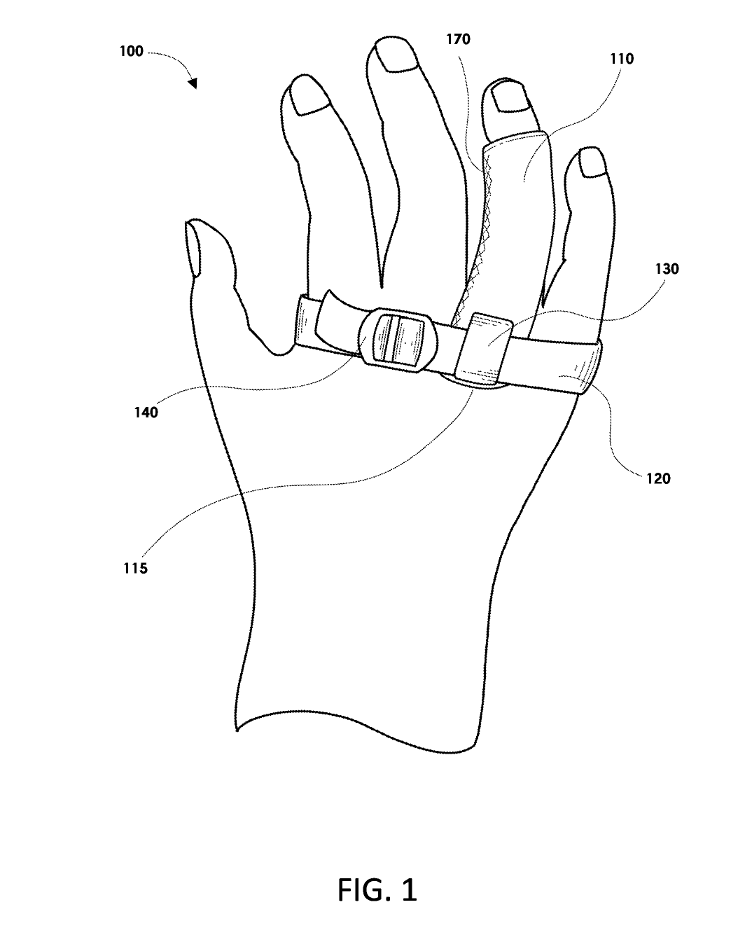

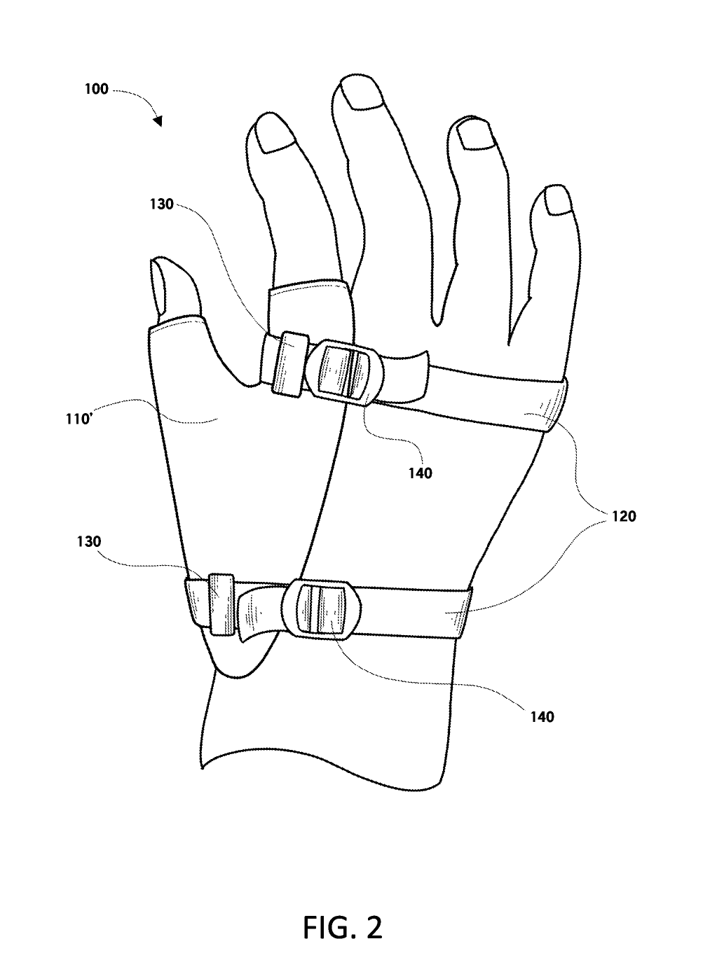

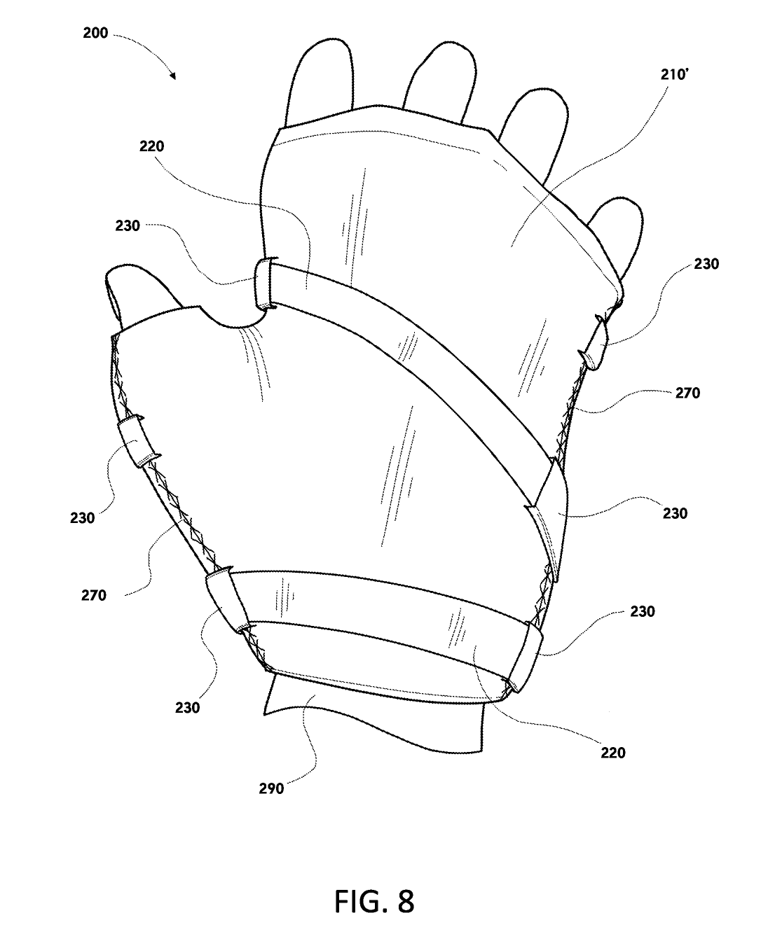

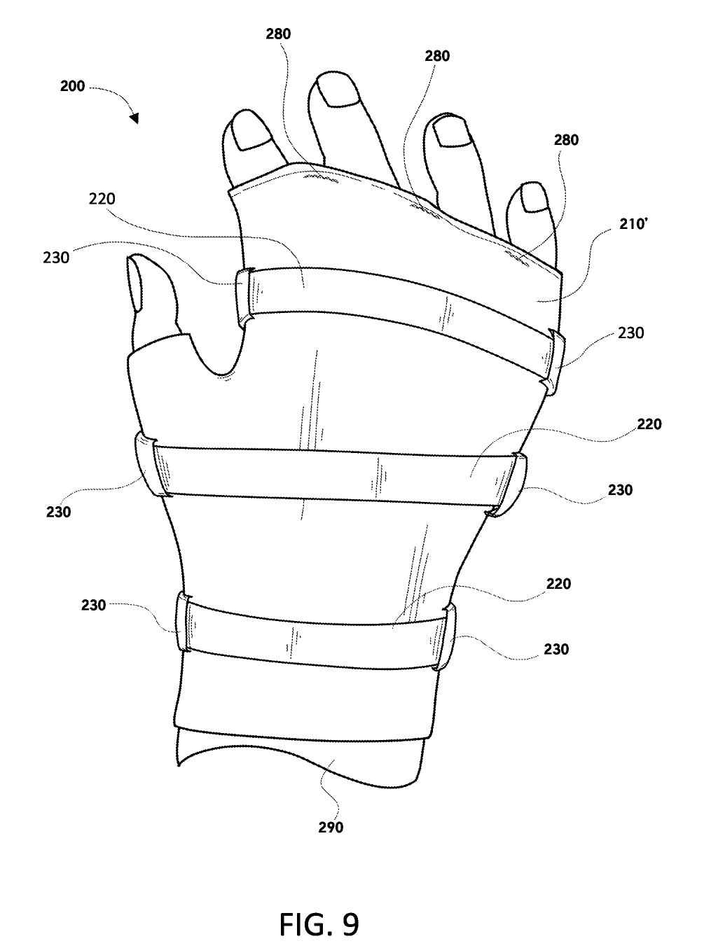

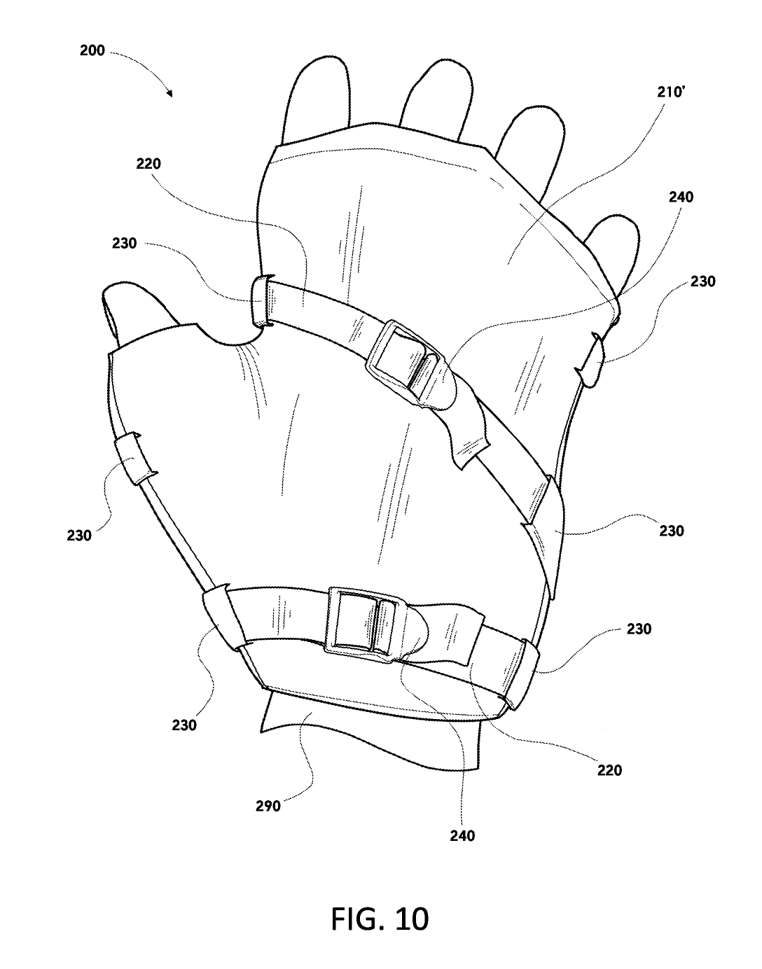

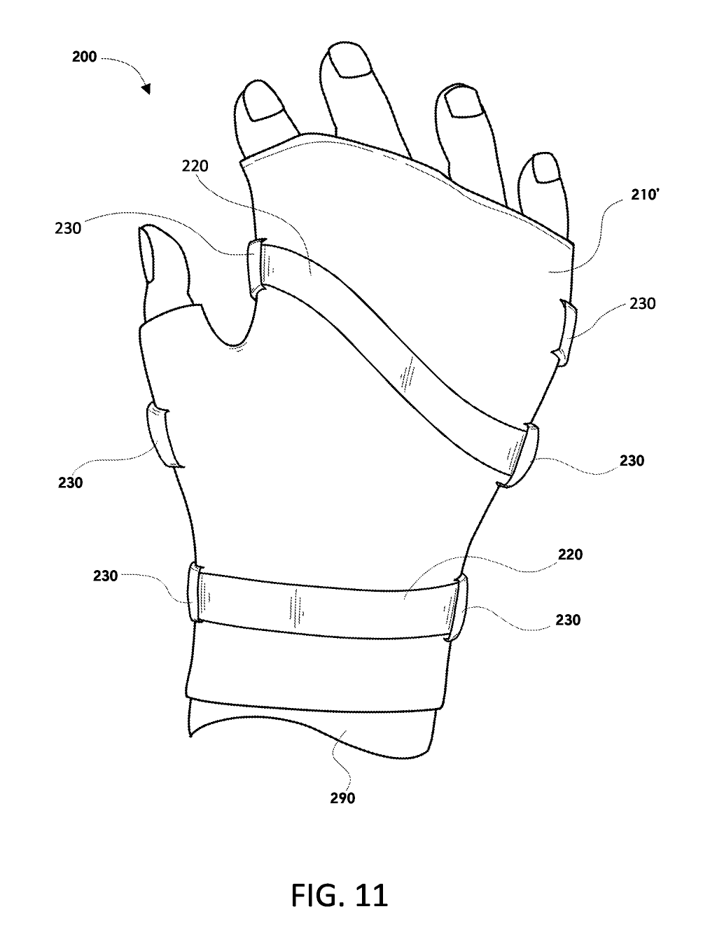

FIGS. 1-11 illustrate therapy garments that may be used with a finger or a hand, according to embodiments of the present invention. Such therapy garments may be referred to, in whole or in part, as therapeutic digit covers. FIG. 1 illustrates a therapy garment 100 including a tubular sleeve 110 having a first opening 115, a strap 120, a guide loop attachment 130, a ladder lock buckle 140, and an elastic insert 170. The user inserts the tubular sleeve 110 onto the finger (e.g. the right ring finger as shown) by inserting the finger through first opening 115. A guide loop attachment 130, which may vary in width, may be stitched or otherwise adhered to the tubular sleeve 110 so as to form a loop through which the strap 120 may be inserted. FIGS. 2-4 illustrate a variation of a tubular sleeve 110′, which includes at a distal end separate openings to receive a user's thumb and index finger, and at a proximal end an opening to cover a portion of the user's hand at the base of the thumb and index finger. Sleeves 110, 110′ are formed of or include a material that is capable of being heated or cooled so as to apply such heating or cooling energy to a user's body, for example for hot or cold therapy. For example, sleeves 110, 110′ may be, or may include (either integrally or via one or more pockets formed therein) a gel pack, flax seed, rice, and/or corn, according to embodiments of the present invention. Sleeves 110, 110′ may be relatively thin so as to include a relatively low profile to permit the user to use his or her hand while the therapy garment is applied. According to other embodiments, sleeves 110, 110′ may be used to apply therapy while the hand is at rest, for example in a “Rest. Ice. Compression. Elevation.” (“RICE”) treatment protocol. Sleeves 110 may be formed of a textile, either synthetic or organic, for example a knit or woven textile, according to embodiments of the present invention.

Each strap 120 may include a ladder lock buckle 140 which permits the strap 120 to be wrapped around the user's hand and attached back onto itself in an easily adjustable manner via the ladder lock buckle 140, according to embodiments of the present invention. Ladder lock buckles 140 are easy to adjust—they may be tightened by simply pulling the free end of the strap 120, and loosened by simply lifting one side of the buckle 140 upwardly. The guide loop attachments 130 may vary in width; for example, a relatively thin guide loop attachment 130 helps to keep the sleeve 110, 110′ in place with respect to a strap 120, while a wider guide loop attachment 130, which may be referred to as a “patch,” permits greater precision in the positioning of the sleeve 110, 110′ with respect to the strap 120 because the strap 120 contacts the guide loop attachment 130 over a greater distance when the guide loop attachment 130 is wider, according to embodiments of the present invention. The guide loop attachment 130 may also be used to assist when putting on the therapy garment.

The arrangement of the straps 120, guide loop attachments 130, and sleeves 110, 110′ permit a particular degree of customization. For example, sleeves 110 and 110′ may be worn at the same time, and the same strap used to secure the top guide loop attachment 130 shown in FIG. 2 may also be used to secure the guide loop attachment 130 shown in FIG. 1, according to embodiments of the present invention. As another alternative, the tubular sleeve 110 of FIG. 1 may be placed on a different finger. As yet another alternative, multiple sleeves 110 may be included, for example in a kit, and the user may decide how many of such sleeves 110 to apply to fingers (e.g. from one to four for each hand), and secure the same strap 120 through the guide loop attachment 130 of each such sleeve 110, according to embodiments of the present invention.

According to embodiments of the present invention, the distal tip of each sleeve 110 is open so as to permit the user to experience tactile sensation at the user's fingertip even while the therapy garment 100 is applied. This feature may also help the user to monitor the status of blood flow or oxygenation to the digit, for example by observing the coloration of the skin at the fingertip. This feature may also permit other devices to be applied to the fingertip even while the therapy garment 100 is applied thereto (e.g. a pulse oximeter), or may permit the user to apply a pinprick to the fingertip to self-administer a blood glucose test, according to embodiments of the present invention. This feature may also provide greater comfort because the fingertips tend to be more temperature sensitive, and by leaving them uncovered by the therapy garment 100 the user may be able to wear the therapy garment 100 for a longer period of time, according to embodiments of the present invention.

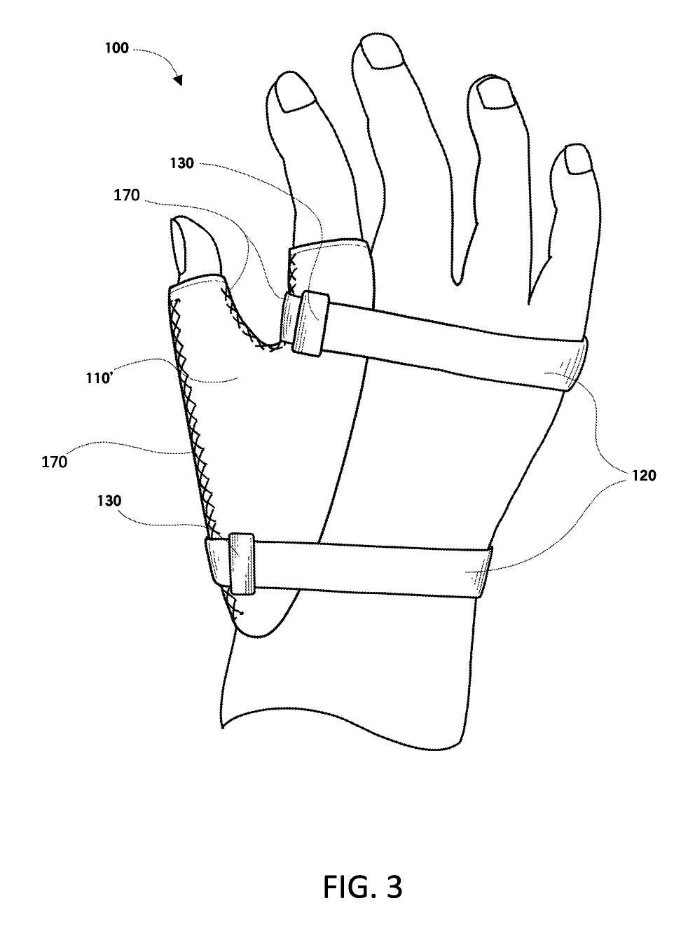

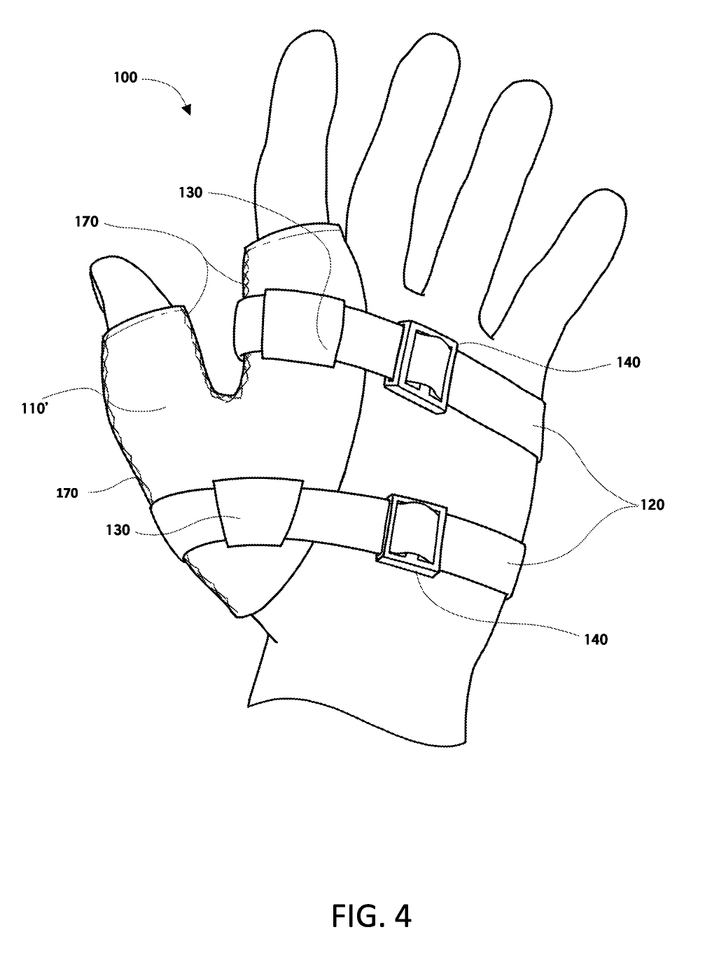

As shown in FIG. 2, the ladder buckle 140 is on the posterior side of the sleeve 110′, according to embodiments of the present invention. As shown in FIGS. 3 and 4, the ladder buckle 140 may be located on an anterior side of sleeve 110′, according to embodiments of the present invention.

A therapeutic digit cover 100 according to an embodiment of the present invention includes a tubular sleeve 110, 110′ for one or more digits, wherein the tubular sleeve 110, 110′ has a first opening 115 and a length and is closed along at least part of the length, as shown in FIG. 1. A thermal pack is incorporated in the tubular sleeve 110, the thermal pack is fully enclosed in the tubular sleeve 100 such that there is no direct contact of the thermal pack with a user's skin when the therapeutic digit cover 100 is worn by the user. The therapeutic digit cover 100 further includes a strap 120 with a closed loop, wherein the closed loop is configured to encircle a palm of the hand between an index finger and a thumb of the user when the therapeutic digit cover 100 is worn by the user. The digit cover 100 may further include at least one guide loop attachment 130 to hold the strap 120 in place on the user's hand.

According to some embodiments, the tubular sleeve 110 is conical. The tubular sleeve 110 may have a pocket configured to accept the thermal pack, which may be, for example, an open pocket, a stitched closed pocket, and/or a pocket of a different color than that of the therapeutic digit cover 100, according to embodiments of the present invention. In some embodiments, the thermal pack comprises one or more of: gel, flax seed, rice, and corn. In some cases, the first opening 115 is a first opening, and the tubular sleeve 110, 110′ has a second opening for a second digit of the hand of the user. According to some embodiments, the tubular sleeve is configured to accept at least one finger of the hand of the user. The tubular sleeve comprises elastic inserts.

According to some embodiments of the present invention, the guide loop attachment 130 is a patch that allows for compression to be administered in specific areas of the user's hand. The guide loop attachment 130 may vary in width, for example from 0.1 inches to 2 inches, according to embodiments of the present invention. The strap 120 may be adjustable, and may, for example, include a ladder lock buckle 140 for adjustment of the strap. In some cases, the tubular sleeve 110, 110′ extends up to a distal phalanx of the digit, as shown in FIG. 1. FIGS. 3-4 show elastic inserts 170 on one or both sides of the tubular sleeve 110′ to permit expansion of the sleeve 110′ during insertion of the fingers, and also provide for greater comfort of the sleeve 110′ when worn, according to embodiments of the present invention.

A method of administering therapy to a digit of a user according to an embodiment of the present invention includes positioning a therapeutic digit cover 100 comprising a tubular sleeve 110 to cover at least one digit, wherein the tubular sleeve 110 has a first opening 115 and is closed along at least part of its length; chilling or heating a thermal pack; securing the therapeutic digit cover 100 with a strap 120, wherein the strap 120 is a closed loop that encircles a palm of a hand between an index finger and a thumb of the user; and adjusting the strap 120 to apply pressure to a desired portion of the therapeutic digit cover 100.

In some cases, adjusting the strap 120 includes tightening and loosening the strap 120 using a ladder lock buckle fastener 140. Adjusting the strap includes looping the strap through one or more guide loop attachments to hold the strap in place, according to embodiments of the present invention. In some cases, the therapeutic digit cover is a first therapeutic digit cover, the method further comprising positioning a second therapeutic digit cover on the hand of the user.

A therapeutic digit cover kit 100′ according to an embodiment of the present invention includes: a first digit cover 110 having a distal end and a proximal end, the proximal end comprising a first guide loop attachment 130; a second digit cover 110 (second digit cover not shown in FIG. 1) having a distal end and a proximal end, the proximal end comprising a second guide loop attachment 130, wherein the first digit cover 110 is independent of the second digit cover 110; a strap 120 configured to form a closed loop, wherein the closed loop is configured to encircle the palm of a hand of a user between an index finger and thumb of the user; wherein the strap 120 is selectively insertable through one or both of the first guide loop 130 and second guide loop attachment 130 for customizing a number of digits covered. The strap 120 may include a ladder lock buckle fastener 140.

According to some embodiments, the therapeutic digit cover kit 100′ further includes a third digit cover 110 having a distal end and a proximal end, the proximal end comprising a third guide loop attachment 130, wherein the third digit cover 110 is independent of the first and second digit covers, and wherein the strap 120 is selectively insertable through one or more of the first, second, and third guide loop attachments 130 for customizing the number of digits covered. According to some embodiment, the therapeutic digit cover kit 100′ further includes a fourth guide loop attachment 110, wherein the fourth digit cover is independent of the first, second, and third digit covers, and wherein the strap 120 is selectively insertable through one or more of the first, second, third, and fourth guide loop attachments for customizing the number of digits covered.

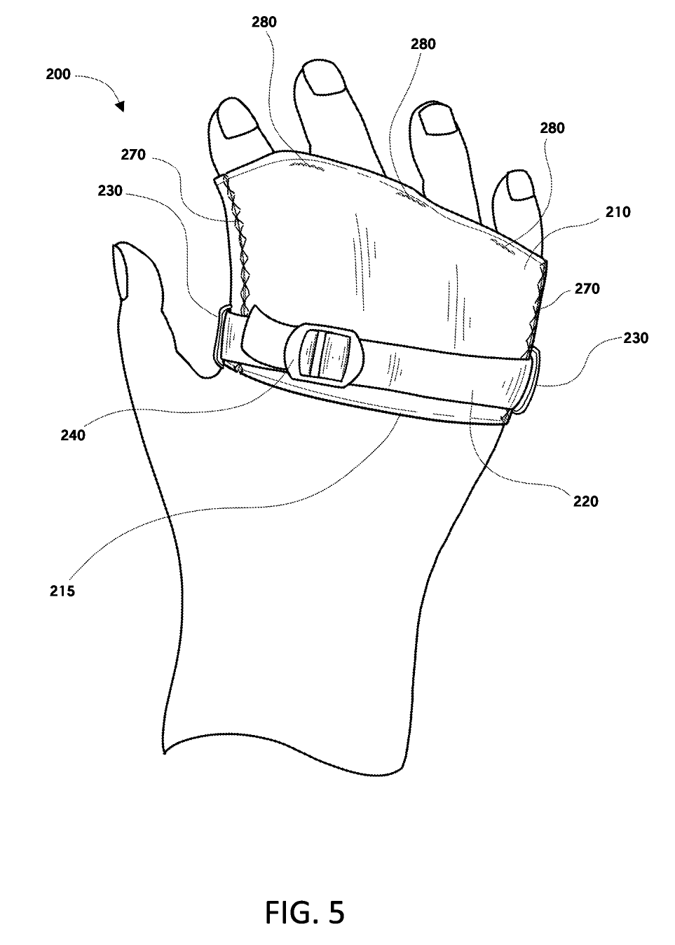

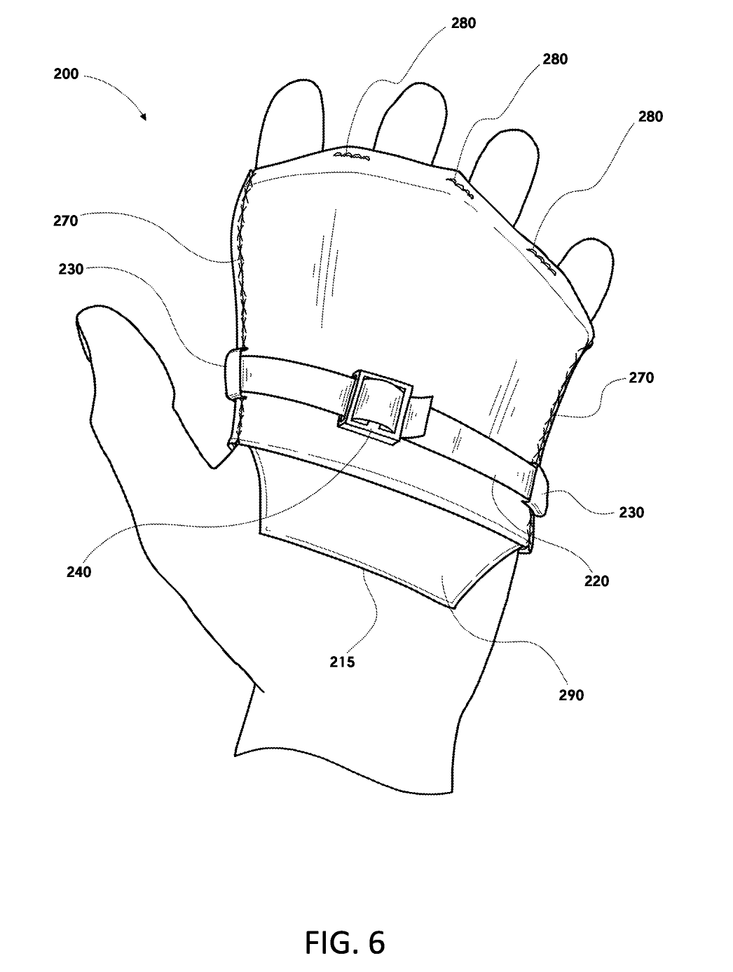

FIGS. 5 and 6 illustrate an alternative hand therapy garment 200, according to embodiments of the present invention. Garment 200 includes a tubular sleeve 210, a strap 220, guide loop attachments 230, a ladder lock buckle 240, elastic inserts 270, stitching 280, and a flap 290, according to embodiments of the present invention. The tubular sleeve 210 may be slid over the fingers of a hand as shown, with the top opening of the sleeve 210 stitched together between each finger for better stability and feel. Elastic inserts 270 on one or both sides of the tubular sleeve 210 permit expansion of the sleeve 210 during insertion of the hand and fingers, and also provide for greater comfort of the sleeve 210 when worn, according to embodiments of the present invention. The guide loop attachments 230 provide placement for the strap 220 secured around the user's hand. As described above, the tubular sleeve 210 may be or have integrated therewith a thermal pack, such as a gel pack, for hot or cold therapy. Alternatively, tubular sleeve 210 may include a pocket; the pocket may open at flap 290 which may extend beyond the pocket like the top flap of an envelope. In some embodiments, the flap 290 extends from the side of the pocket that goes against a user's skin, so that the flap 290, when folded back into the pocket and/or under the strap 220 does not form a double insulating layer between the thermal pack and the user's skin, according to embodiments of the present invention. The buckle 240 of the strap 220 may be located on either the anterior or posterior hand surface, according to embodiments of the present invention.

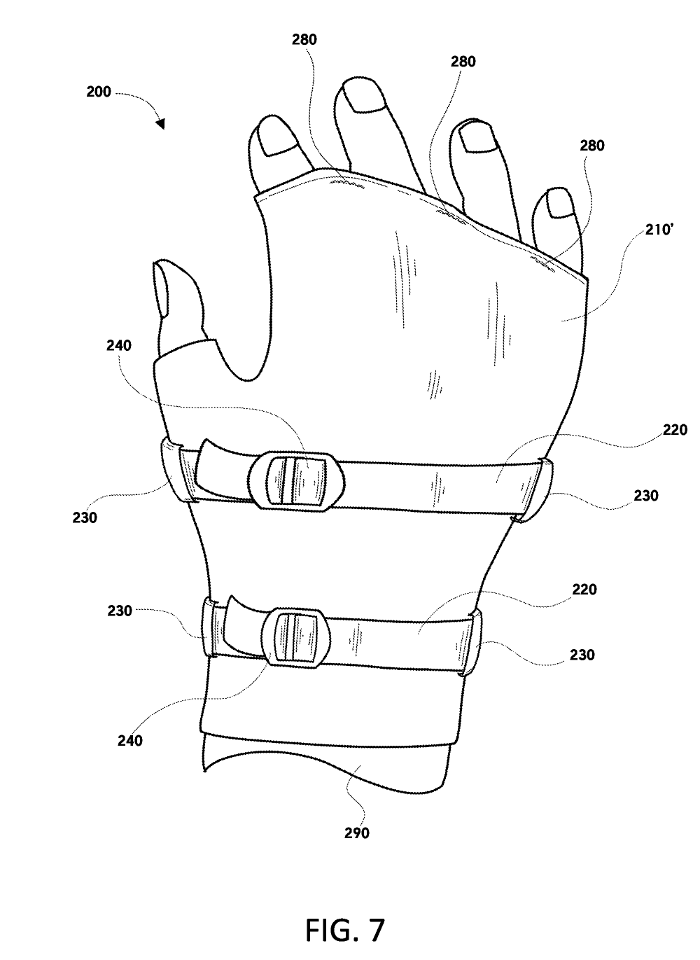

FIGS. 7-11 illustrate an alternative hand therapy garment 200 which forms more of a full mitt about the hand, according to embodiments of the present invention. Garment 200 in these depictions includes a tubular sleeve 210′, a strap 220, guide loop attachments 230, a ladder lock buckle 240, elastic inserts 270, stitching 280, and a flap 290, according to embodiments of the present invention. The tubular sleeve 210′ may be slid over the fingers and thumb of a hand as shown, with the top opening of the sleeve 210′ stitched together between each finger with stitching 280 for better stability and feel. The guide loop attachments 230 provide placement for the strap 220 secured around the user's hand. As described above, the tubular sleeve 210′ may be or have integrated therewith a thermal pack, such as a gel pack, for hot or cold therapy. Alternatively, tubular sleeve 210′ may include a pocket; the pocket may open at flap 290 which may extend beyond the pocket like the top flap of an envelope. In some embodiments, the flap 290 extends from the side of the pocket that goes against a user's skin, so that the flap 290, when folded back into the pocket and/or under the strap 220 does not form a double insulating layer between the thermal pack and the user's skin, according to embodiments of the present invention. In some embodiments, the tubular sleeve 210′ and/or its shape or structuring, holds the user's thumb in the abducted position, allowing for better temperature treatment to the thenar eminence, which is the fleshy area at the base of the thumb into the palm.

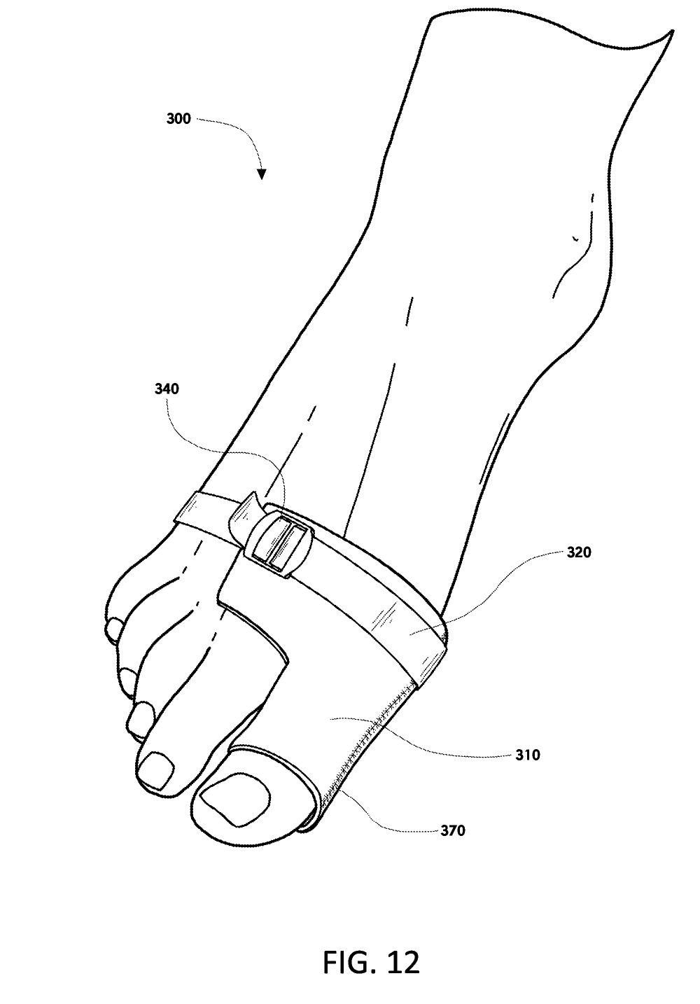

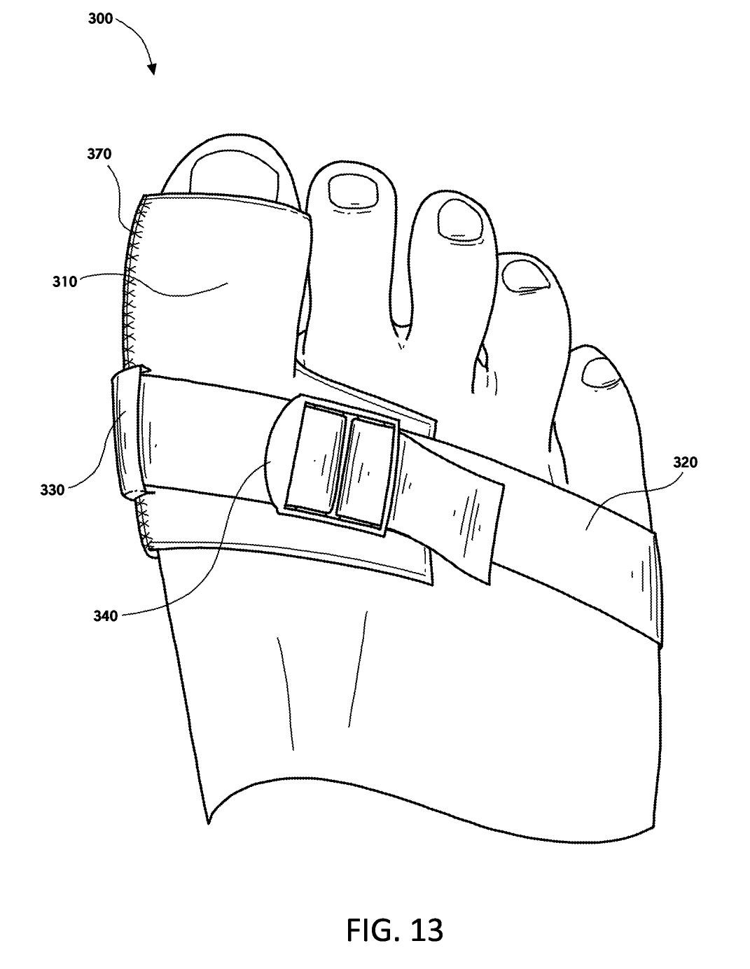

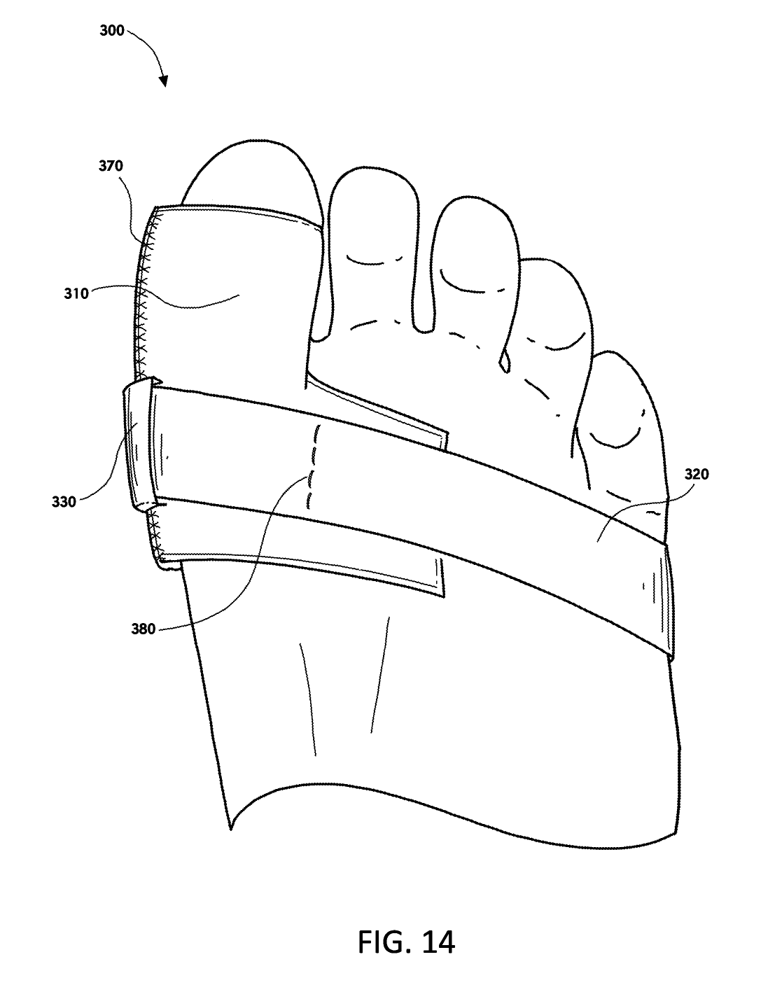

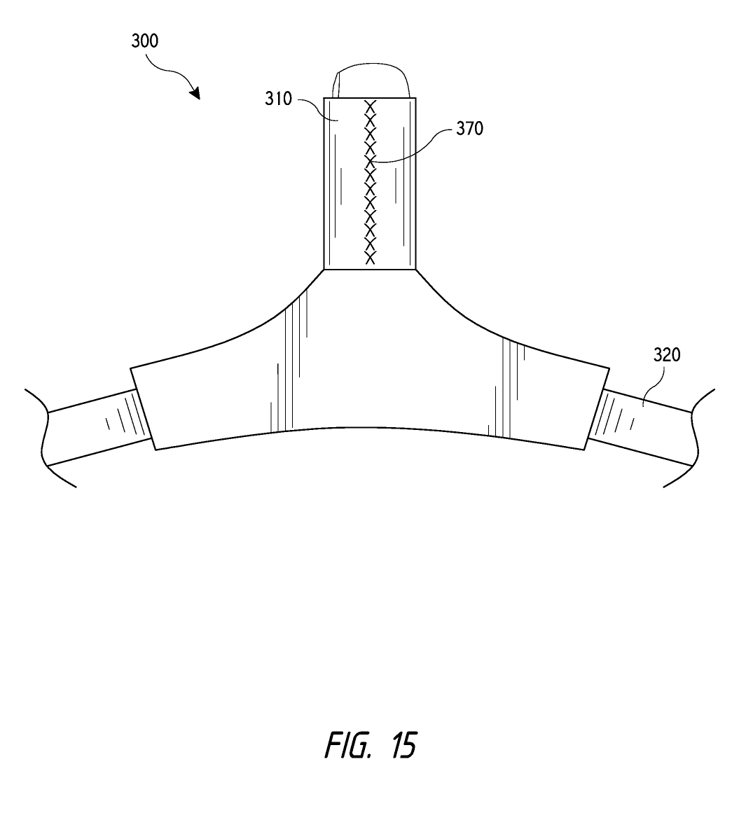

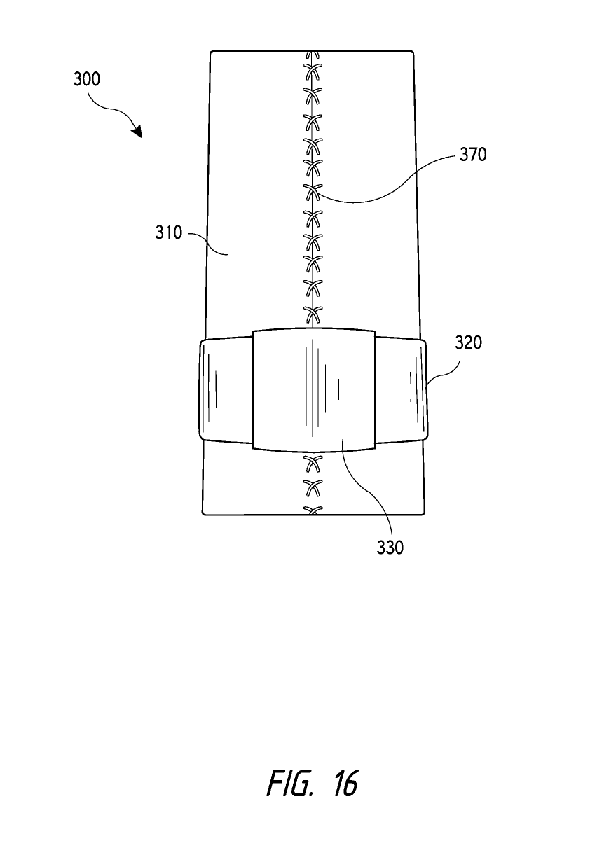

FIGS. 12-16 illustrate a great toe therapy garment 300, according to embodiments of the present invention. Garment 300 in these depictions includes a tubular sleeve 310, a strap 320, guide loop attachments 330, ladder lock buckles 340, elastic inserts 370, and stitching 380, according to embodiments of the present invention. The tubular sleeve 310 may be slid over the great toe as shown, with the top opening of the sleeve 310 optionally being open at its distal end, similar to sleeve 110. The guide loop attachments 330 provide placement for the strap 320 secured around the user's foot. The guide loop attachment 330 may also be used to assist when putting on the therapy garment. As described above, the tubular sleeve 310 may be or have integrated therewith a thermal pack, such as a gel pack, for hot or cold therapy. Alternatively, tubular sleeve 310 may include a pocket to receive a thermal pack. The adjustment buckle 340 may be located on a top side of the strap 320 to permit the user to easily access the buckle 340 with a hand for adjusting, according to embodiments of the present invention. The strap 320 is secured to the tubular sleeve 310 with stitching 380 to keep the strap 320 in place. Elastic inserts 370 on one or both sides of the tubular sleeve 310 permit expansion of the sleeve 310 during insertion of the toe, and also provide for greater comfort of the sleeve 310 when worn, according to embodiments of the present invention.

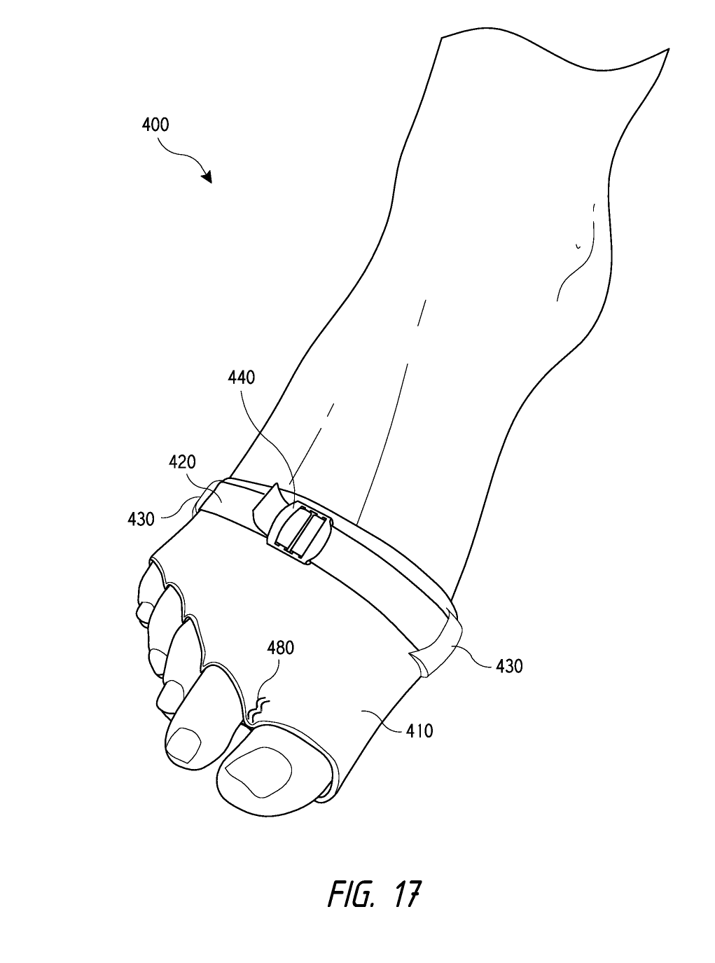

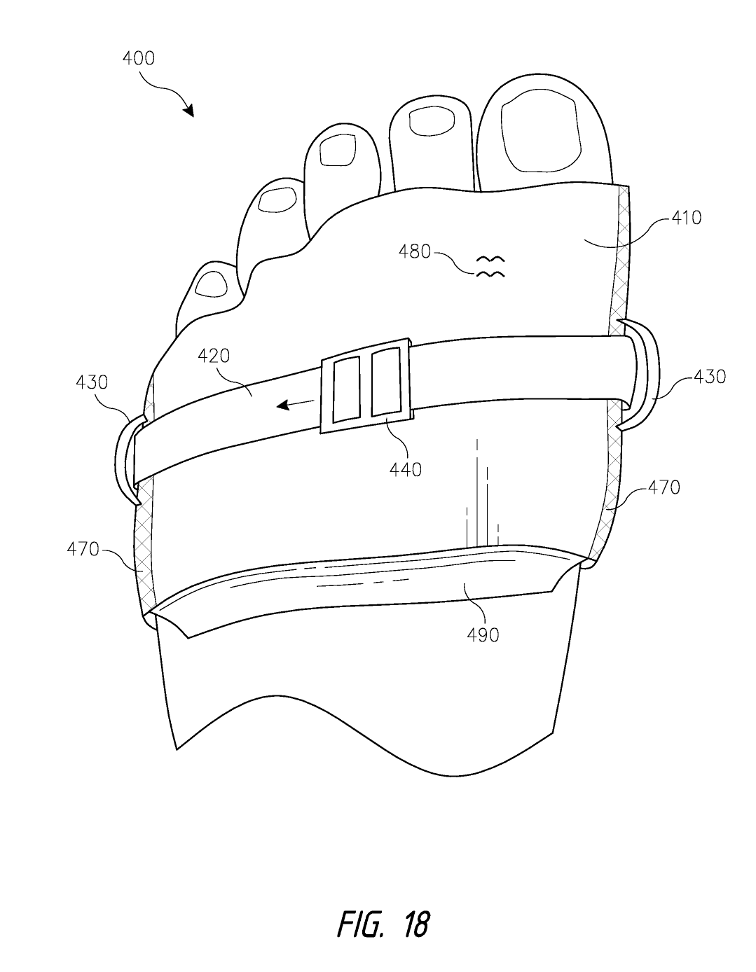

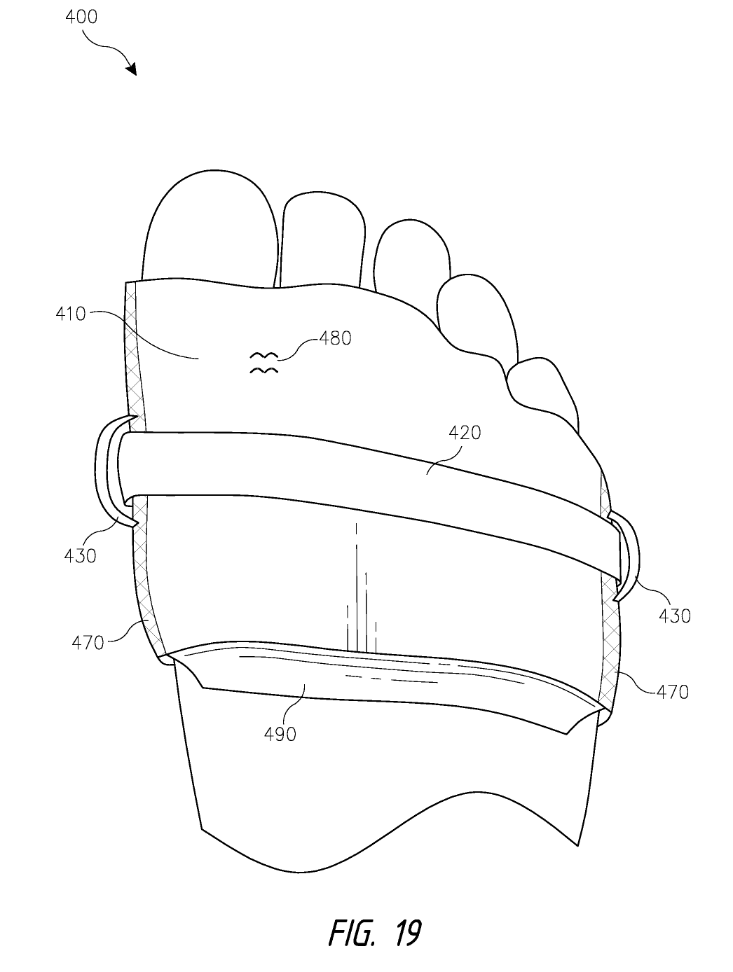

FIGS. 17-19 illustrate a pedi therapy garment 400, according to embodiments of the present invention. Garment 400 in these depictions includes a tubular sleeve 410, a strap 420, guide loop attachments 430, ladder lock buckles 440, elastic inserts 470, stitching 480, and a flap 490, according to embodiments of the present invention. The tubular sleeve 410 may be slid over the end of the foot as shown, with the top opening of the sleeve 410 optionally being open at its distal end, similar to sleeve 110. The guide loop attachments 430 provide placement for the strap 420 secured around the user's foot. As described above, the tubular sleeve 410 may be or have integrated therewith a thermal pack, such as a gel pack, for hot or cold therapy. Alternatively, tubular sleeve 410 may include a pocket to receive a thermal pack; the pocket may open at flap 490 which may extend beyond the pocket like the top flap of an envelope. In some embodiments, the flap 490 extends from the side of the pocket that goes against a user's skin, so that the flap 490, when folded back into the pocket and/or under the strap 420 does not form a double insulating layer between the thermal pack and the user's skin, according to embodiments of the present invention.

The adjustment buckle 440 may be located on a top side of the strap 420 to permit the user to easily access the buckle 440 with a hand for adjusting, according to embodiments of the present invention. Elastic inserts 470 on one or both sides of the tubular sleeve 410 permit expansion of the sleeve 410 during insertion of the foot, and also provide for greater comfort of the sleeve 410 when worn, according to embodiments of the present invention. Stitching 480 may be placed to secure opposite ends of the distal opening of the tubular sleeve 410; for example, such stitching may be place between the big toe and the adjacent toe only (as shown in FIGS. 17-19), or alternatively stitching 480 may be placed between two or more or all of the toes, according to embodiments of the present invention.

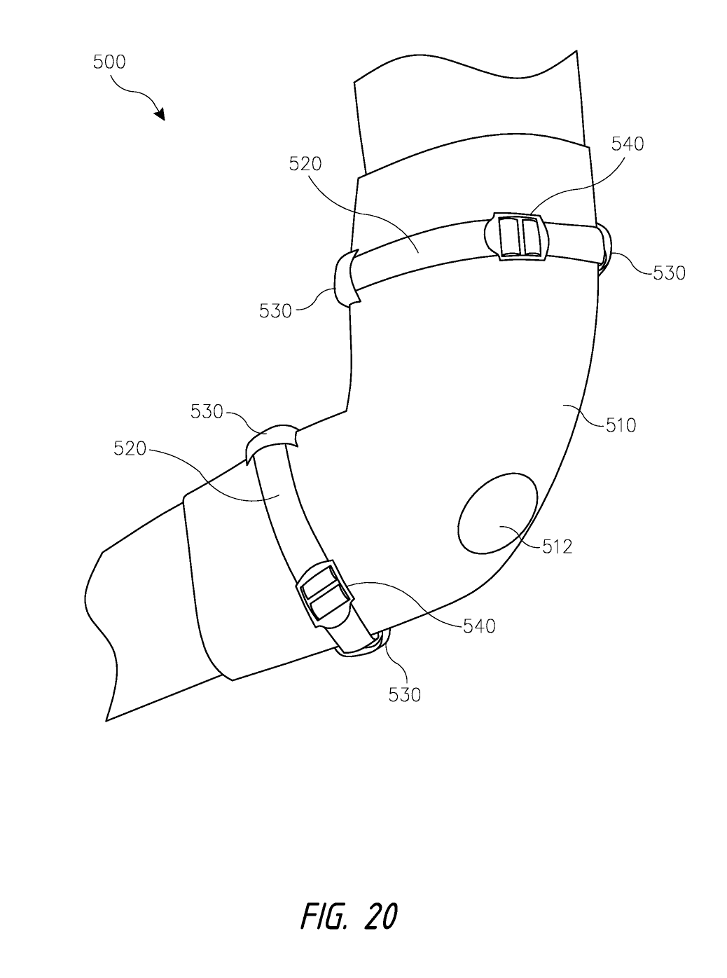

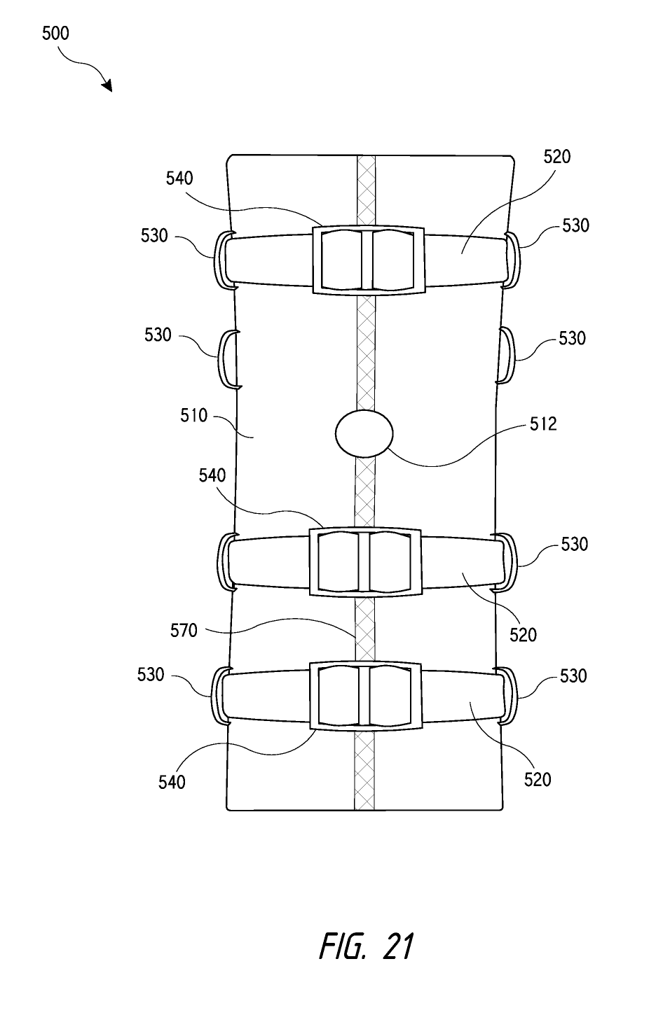

FIGS. 20-22 illustrate an elbow therapy garment 500, according to embodiments of the present invention. The elbow therapy garment 500 can be used in the area of the elbow as well as the areas proximal and distal to the elbow. Garment 500 in these depictions includes a tubular sleeve 510, straps 520, guide loop attachments 530, ladder lock buckles 540, elastic inserts 570, and a flap 590, according to embodiments of the present invention. The tubular sleeve 510 may be slid over the end of the arm as shown. The guide loop attachments 530 provide placement for the straps 520 secured around the user's arm. As described above, the tubular sleeve 510 may be or have integrated therewith a thermal pack, such as a gel pack, for hot or cold therapy. Alternatively, tubular sleeve 510 may include a pocket to receive a thermal pack; the pocket may open at flap 590 which may extend beyond the pocket like the top flap of an envelope. In some embodiments, the flap 590 extends from the side of the pocket that goes against a user's skin, so that the flap 590, when folded back into the pocket and/or under the strap 520 does not form a double insulating layer between the thermal pack and the user's skin, according to embodiments of the present invention.

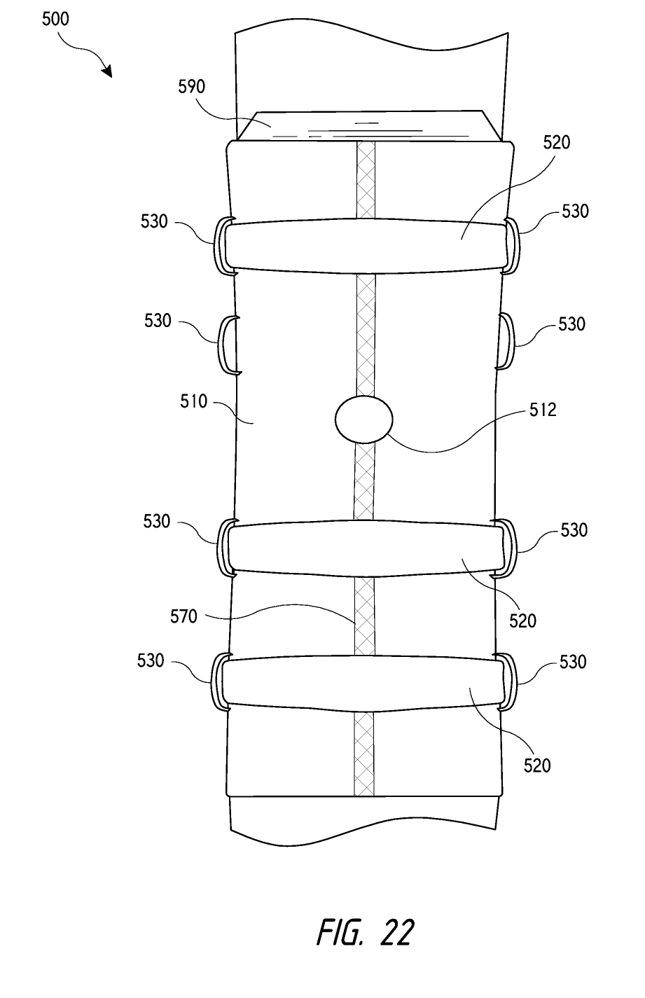

Elastic inserts 570 on one or both sides of the tubular sleeve 510 permit expansion of the sleeve 510 during insertion of the arm, and also provide for greater comfort of the sleeve 510 when worn, according to embodiments of the present invention. As shown in FIG. 22, the elastic insert 570 may be located on one side of the tubular sleeve 510, with a single pocket for a thermal pack wrapping around the front of the garment 500; this wrap-around effect provides for better therapeutic thermal application compared to the user of multiple thermal packs, or the use of flat external thermal packs, according to embodiments of the present invention.

Elbow therapy garment 500 may also include an aperture 512, which may be circular or ovular, according to embodiments of the present invention. Aperture 512, whether located on one or both of the anterior side and the posterior side, serves to create a zone over which the thermal therapy is not applied. This may be desirable when, for example, thermal therapy is intended to be applied to the muscle or other tissue surrounding a joint, for example the elbow joint, without applying such thermal therapy to the (front and/or back of the) joint itself. For example, in some individuals a cold pack may effectively treat swelling in the muscles surrounding the elbow, but making the joint itself colder may increase a risk of injury of the joint. The aperture 512 may further serve to permit the sleeve 510 to stretch more easily, and/or to more easily accommodate an irregular shape of a bending joint such as an elbow or knee (as described below). The aperture 512 may further serve as a gripping or grasping point, in addition to the guide loop attachments 530, according to embodiments of the present invention.

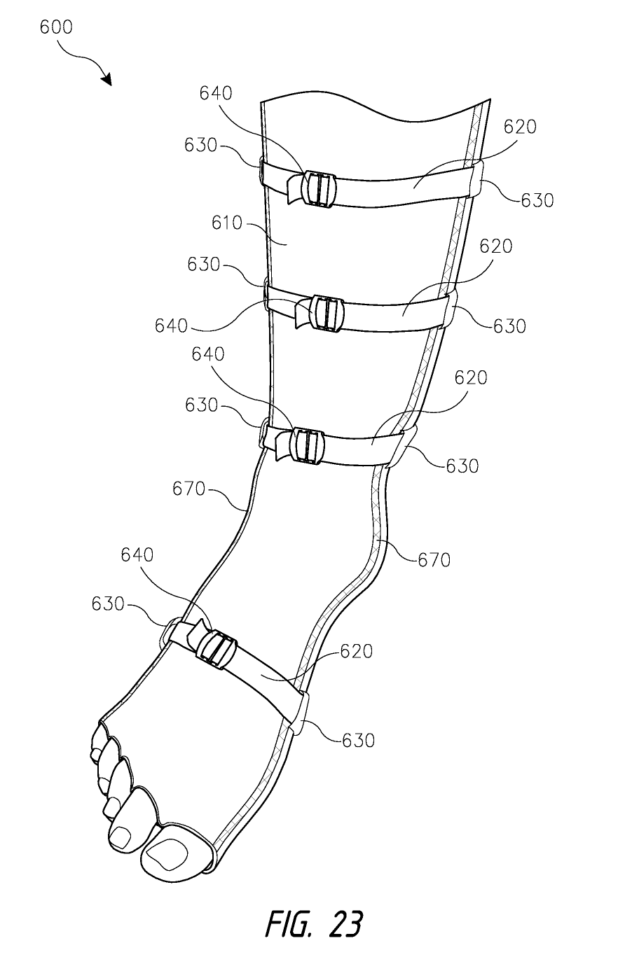

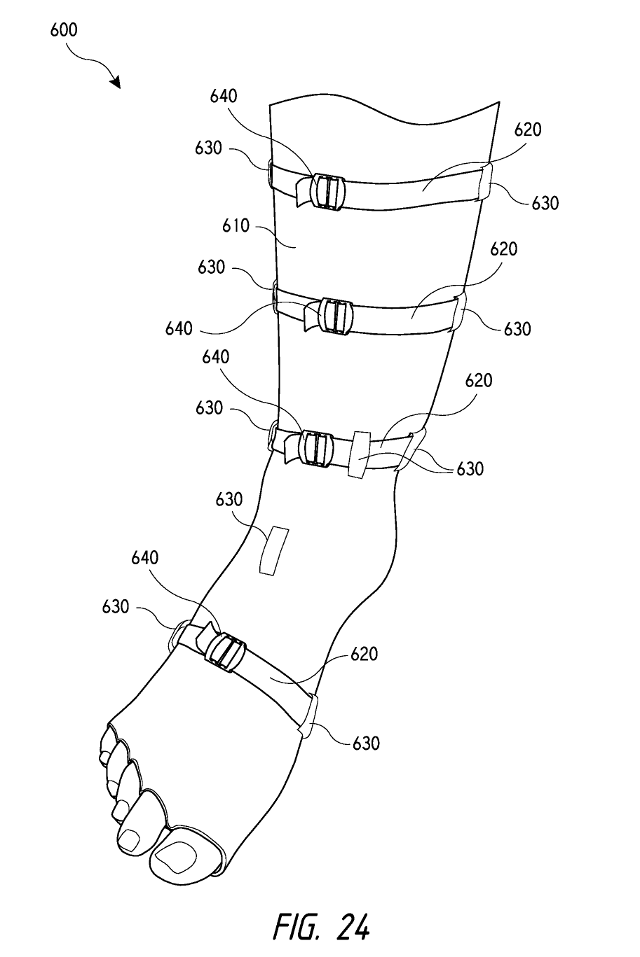

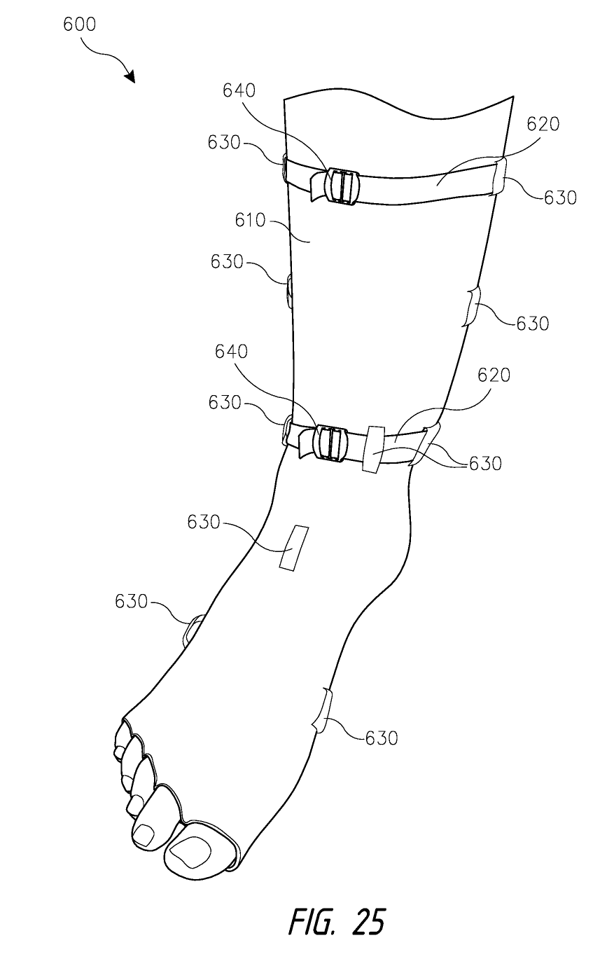

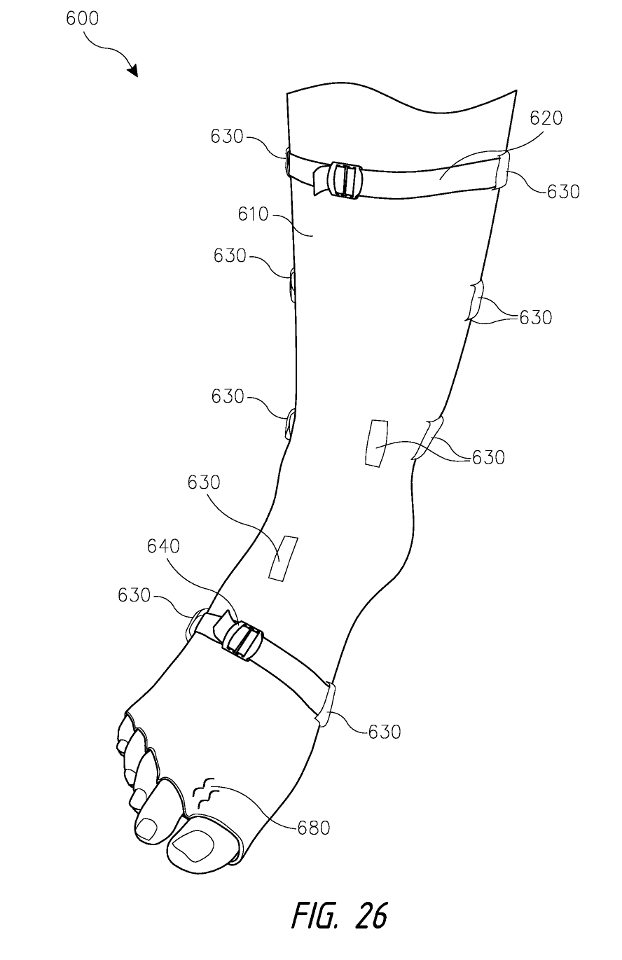

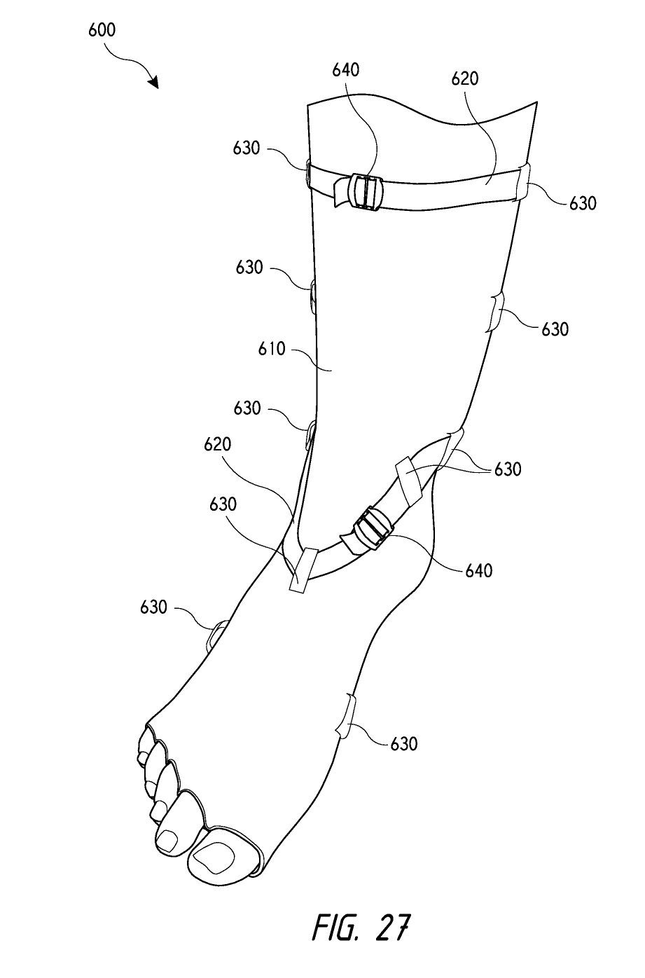

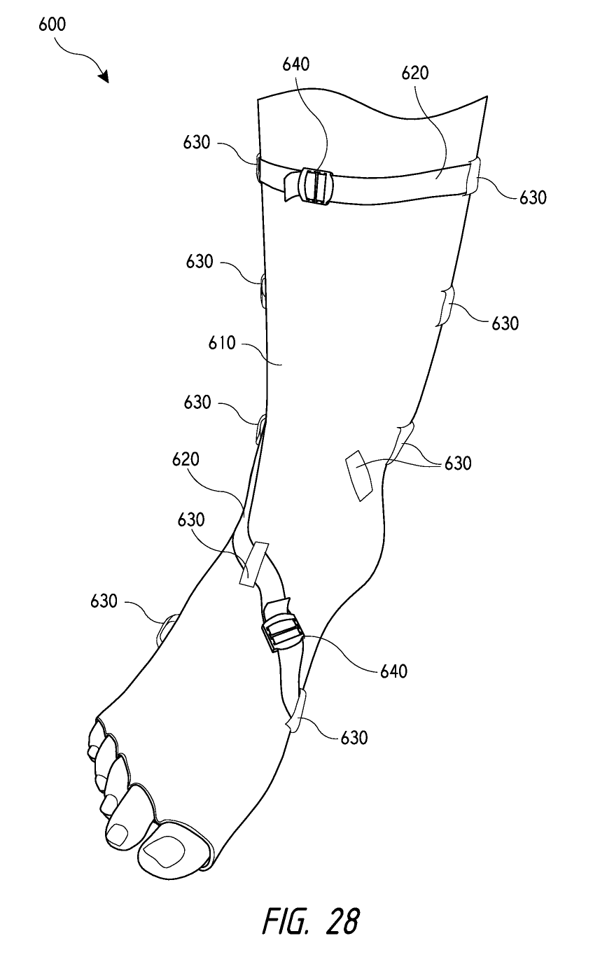

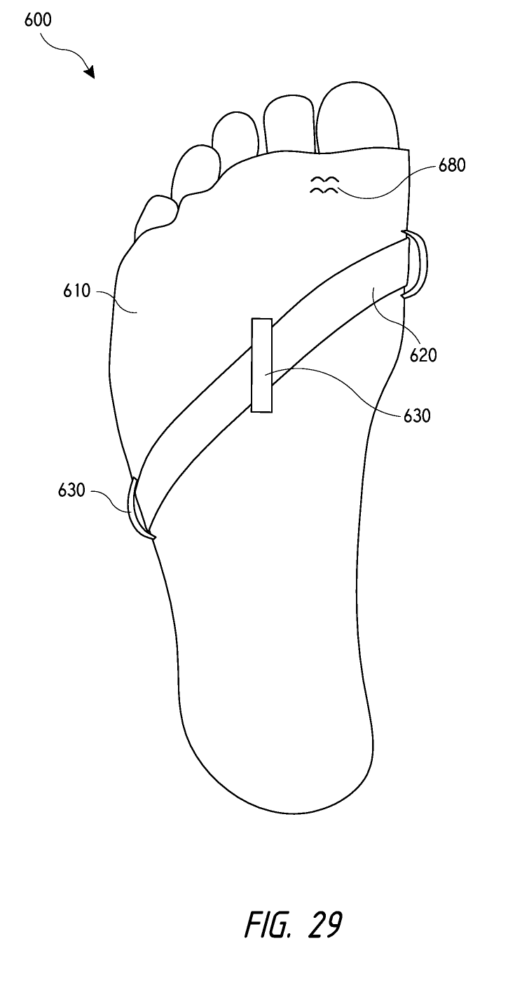

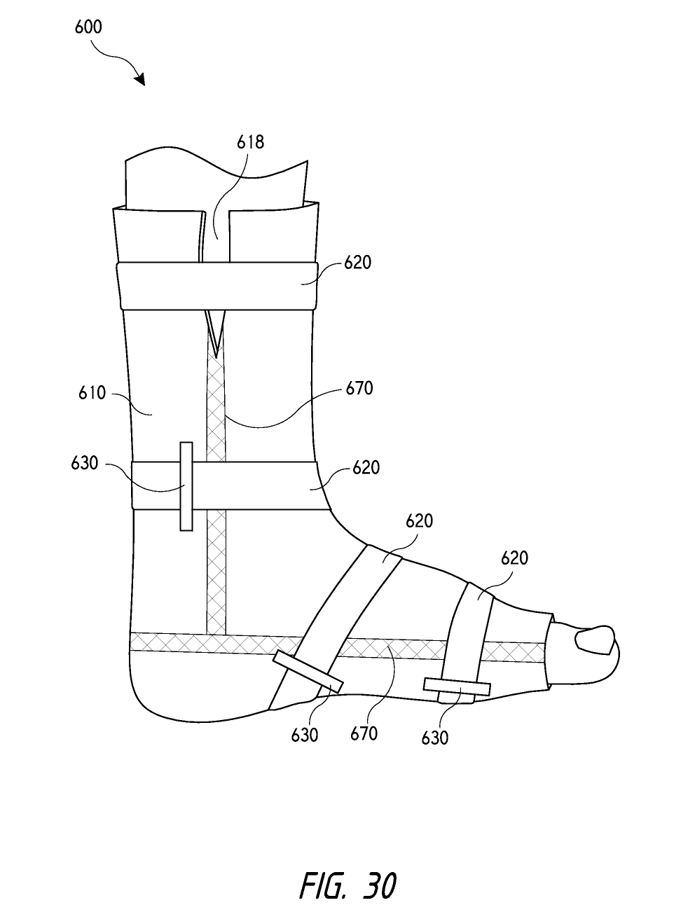

FIGS. 23-30 illustrate therapy sock 600 for a foot, according to embodiments of the present invention. Garment 600 in these depictions includes a tubular sleeve 610, an accommodation slit 618, straps 620, guide loop attachments 630, ladder lock buckles 640, and elastic inserts 670, according to embodiments of the present invention. The tubular sleeve 610 may be slid over the end of the foot as shown. The guide loop attachments 630 provide placement for the straps 620 secured around the user's foot. The varied placement of guide loop attachments 630 permits a custom number of straps 620 to be used, as well as custom placement of those straps 620 according to numerous possible combinations and placements as desired to enhance therapy or attachment strength, according to embodiments of the present invention. FIGS. 23-30 show examples of different strap 620 placements. As described above, the tubular sleeve 610 may be or have integrated therewith a thermal pack, such as a gel pack, for hot or cold therapy. Alternatively, tubular sleeve 610 may include a pocket to receive a thermal pack. While FIG. 23 illustrates elastic inserts 670, one of ordinary skill in the art will appreciate, based on the present disclosure, that such elastic inserts 670 may be included in similar or different locations in the sock examples depicted in FIGS. 24-29, according to embodiments. FIG. 30 also illustrates elastic inserts 670.

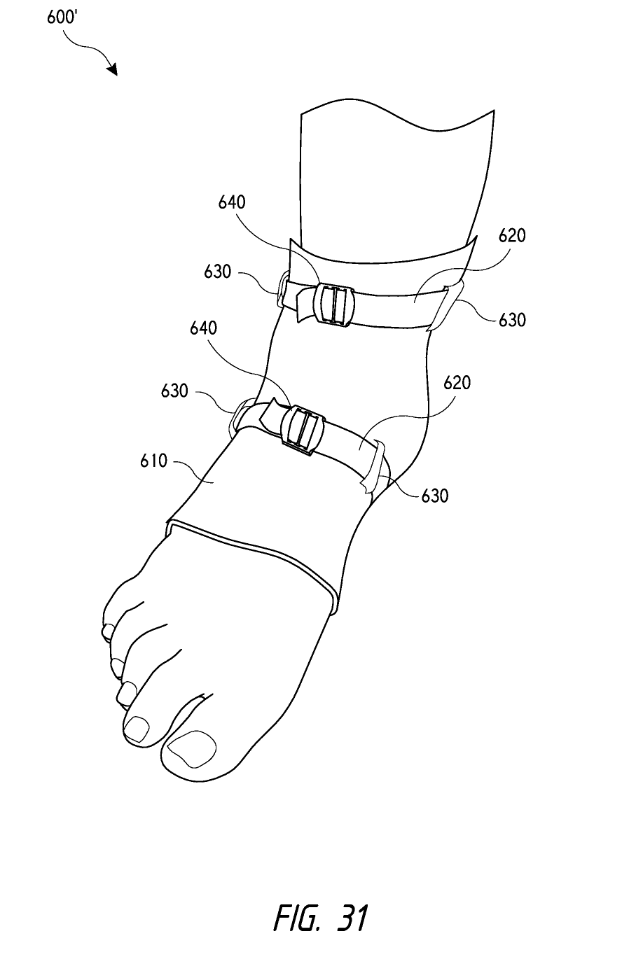

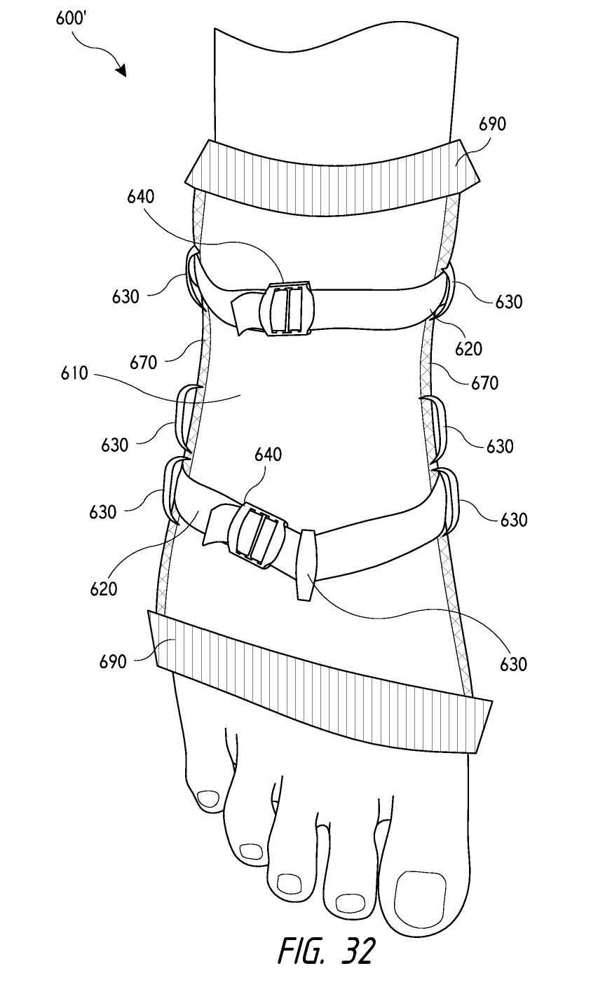

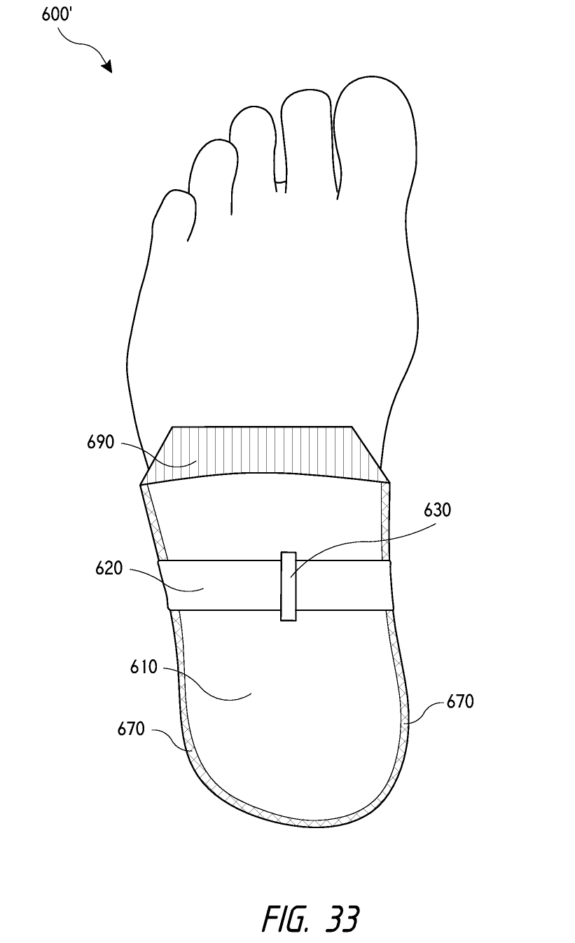

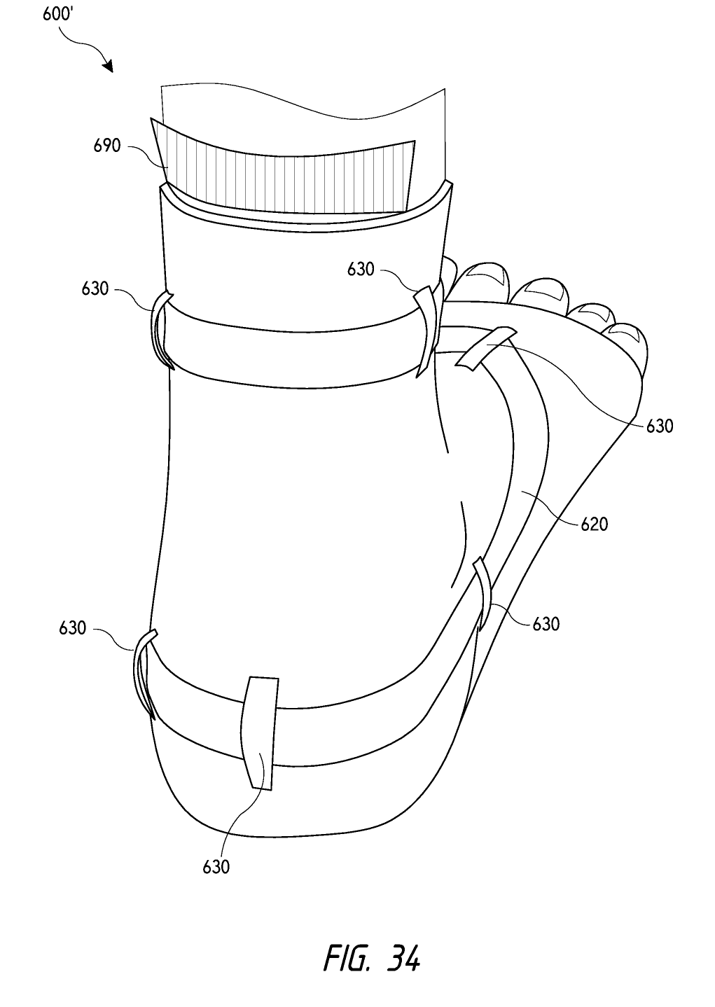

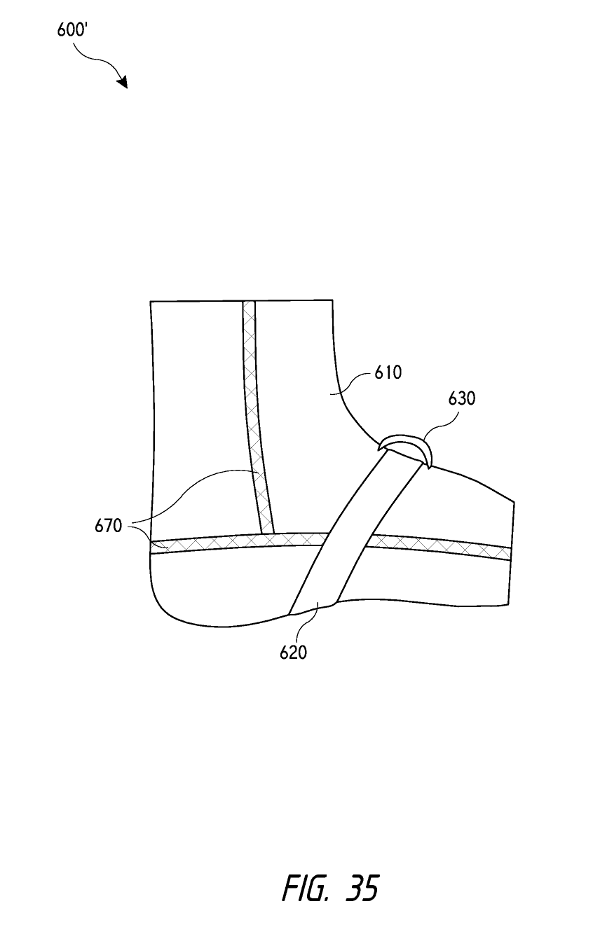

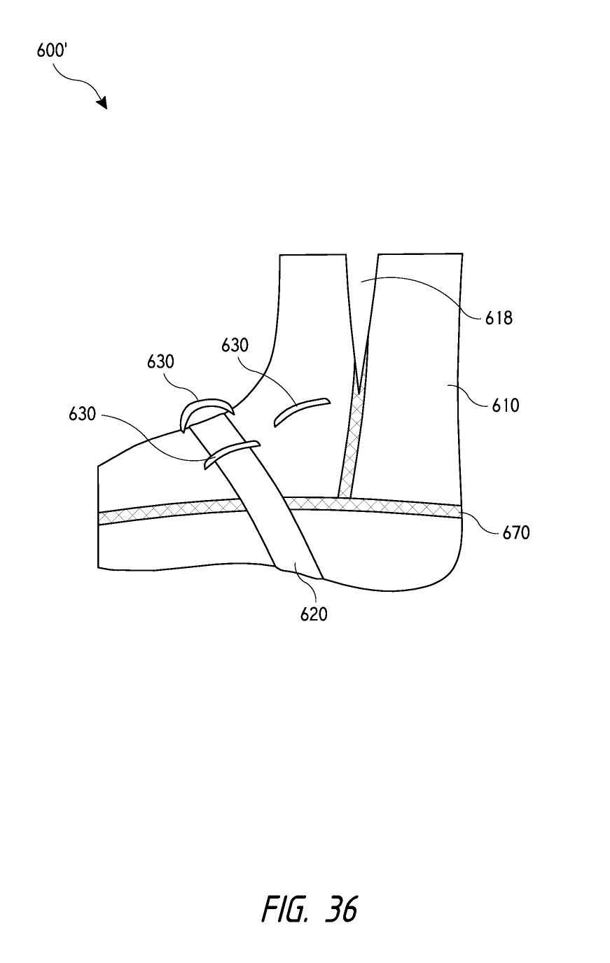

FIGS. 31-36 illustrate an ankle therapy garment 600′, which is essentially a therapy sock similar to that of FIGS. 23-30 but which has been shortened in overall length to focus thermal treatment at the ankle, according to embodiments of the present invention. Garment 600′ in these depictions includes a tubular sleeve 610, an accommodation slit 618, straps 620, guide loop attachments 630, ladder lock buckles 640, elastic inserts 670, and stitching 680, according to embodiments of the present invention. The tubular sleeve 610 may be slid over the end of the foot as shown. The guide loop attachments 630 provide placement for the straps 620 secured around the user's foot. The varied placement of guide loop attachments 630 permits a custom number of straps 620 to be used, as well as custom placement of those straps 620 according to numerous possible combinations and placements as desired to enhance therapy or attachment strength, according to embodiments of the present invention. FIGS. 31-36 show examples of different strap 620 placements. As described above, the tubular sleeve 610 may be or have integrated therewith a thermal pack, such as a gel pack, for hot or cold therapy. Alternatively, tubular sleeve 610 may include a pocket to receive a thermal pack.

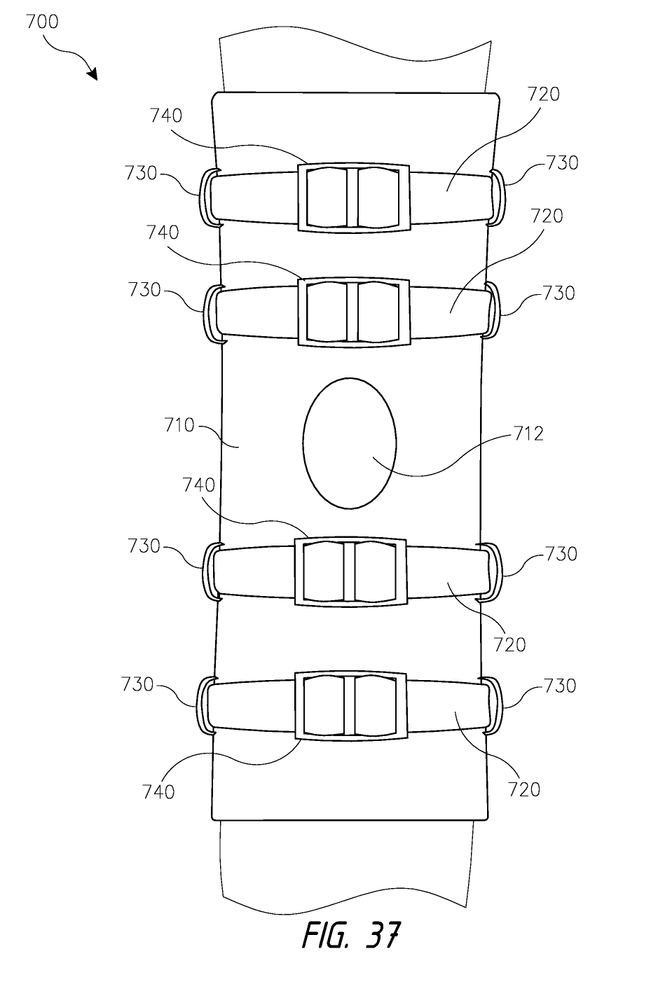

FIGS. 37-39 illustrate a knee therapy garment 700, according to embodiments of the present invention. The knee therapy garment 700 can be used in the area of the knee, as well as the thigh or the calf. Garment 700 in these depictions includes a tubular sleeve 710, straps 720, guide loop attachments 730, ladder lock buckles 740, elastic inserts 770, and flaps 790 according to embodiments of the present invention. The tubular sleeve 710 may be slid over the end of the foot and onto the knee. The guide loop attachments 730 provide placement for the straps 720 secured around the user's knee. The varied placement of guide loop attachments 730 permits a custom number of straps 720 to be used, as well as custom placement of those straps 720 according to numerous possible combinations and placements as desired to enhance therapy or attachment strength, according to embodiments of the present invention. FIGS. 37-39 show examples of different strap 720 placements. As described above, the tubular sleeve 710 may be or have integrated therewith a thermal pack, such as a gel pack, for hot or cold therapy. Alternatively, tubular sleeve 710 may include a pocket to receive a thermal pack; the pocket may open at flap 790 which may extend beyond the pocket like the top flap of an envelope. In some embodiments, the flap 790 extends from the side of the pocket that goes against a user's skin, so that the flap 790, when folded back into the pocket and/or under the strap 720 does not form a double insulating layer between the thermal pack and the user's skin, according to embodiments of the present invention.

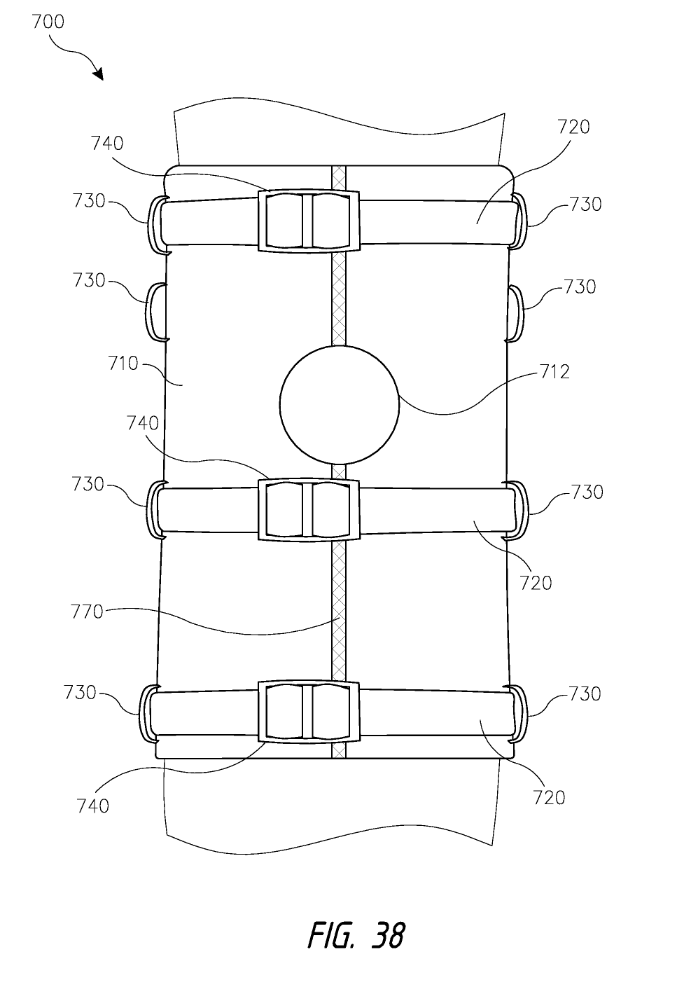

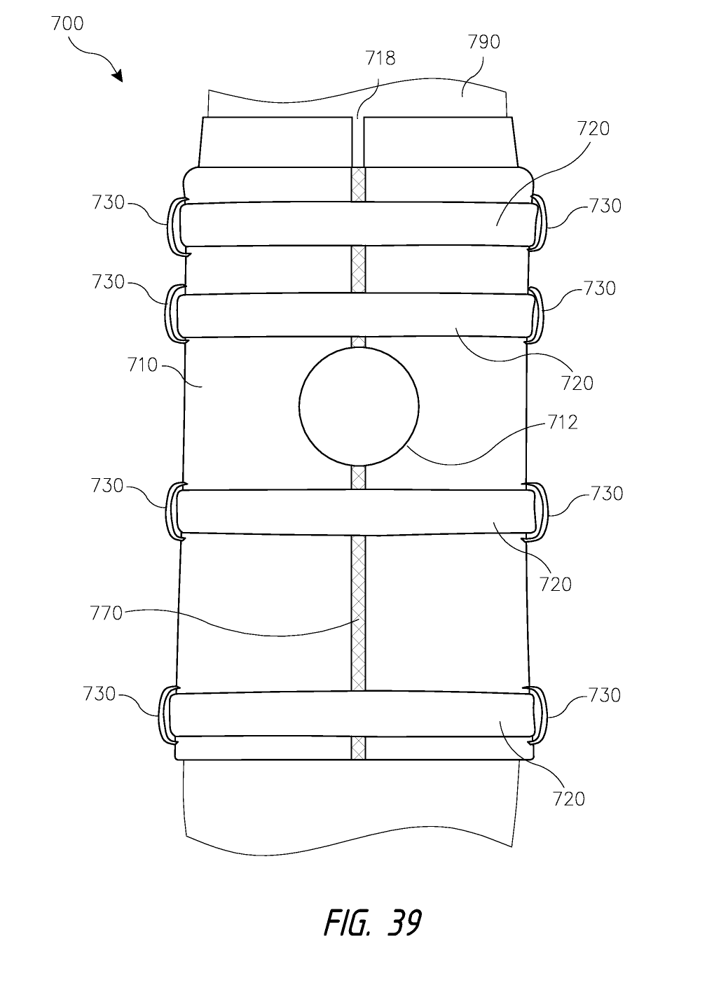

Elastic inserts 770 on one or both sides of the tubular sleeve 710 permit expansion of the sleeve 710 during insertion of the leg, and also provide for greater comfort of the sleeve 710 when worn, according to embodiments of the present invention. As shown in FIGS. 38-39, the elastic insert 770 may be located on one side of the tubular sleeve 710, with a single pocket for a thermal pack wrapping around the front of the garment 700; this wrap-around effect provides for better therapeutic thermal application compared to the user of multiple thermal packs, or the use of flat external thermal packs, according to embodiments of the present invention.

Knee therapy garment 700 may also include an aperture 712, which may be circular or ovular, according to embodiments of the present invention. Aperture 712, whether located on one or both of the anterior side and the posterior side, serves to create a zone over which the thermal therapy is not applied. This may be desirable when, for example, thermal therapy is intended to be applied to the muscle or other tissue surrounding a joint, for example the knee joint, without applying such thermal therapy to the (front and/or back of the) joint itself. For example, in some individuals a cold pack may effectively treat swelling in the muscles surrounding the knee, but making the joint itself colder may increase a risk of injury of the joint. The aperture 712 may further serve to permit the sleeve 710 to stretch more easily, and/or to more easily accommodate an irregular shape of a bending joint such as an elbow or knee (as described below). The aperture 712 may further serve as a gripping or grasping point, in addition to the guide loop attachments 730, according to embodiments of the present invention. In some embodiments, the flap 790 may include a slit 718 for ease of use; the slit may be aligned, in some cases, with the elastic insert 770.

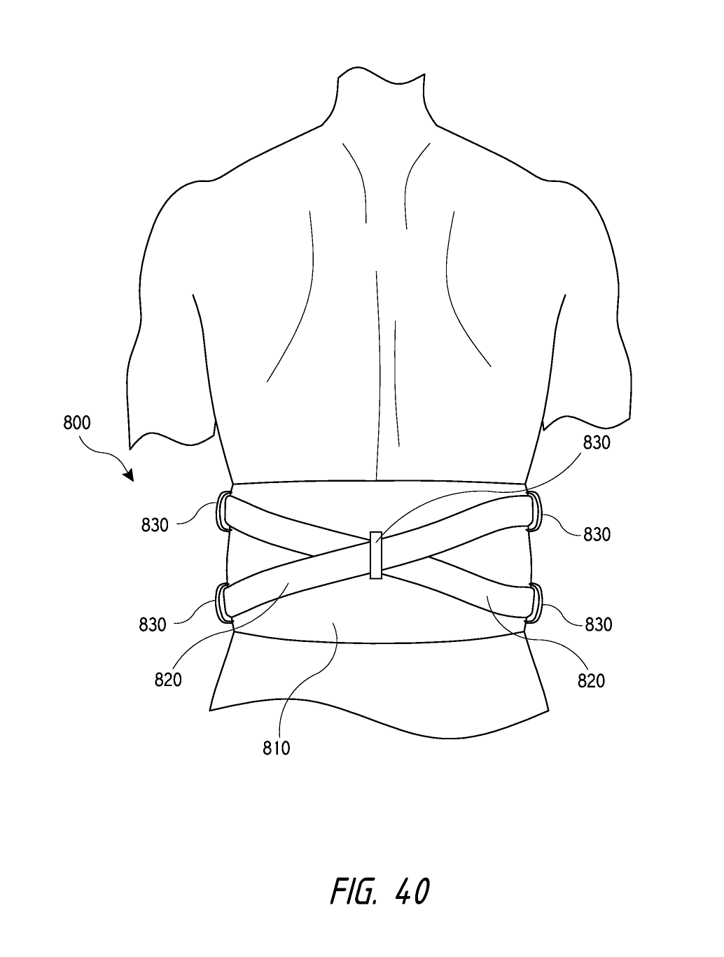

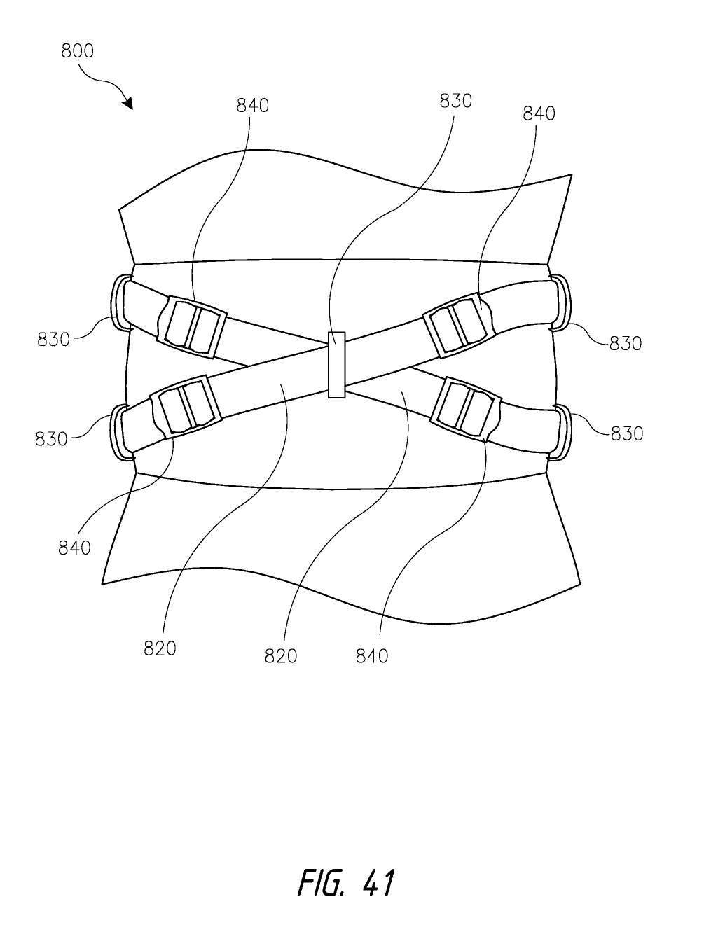

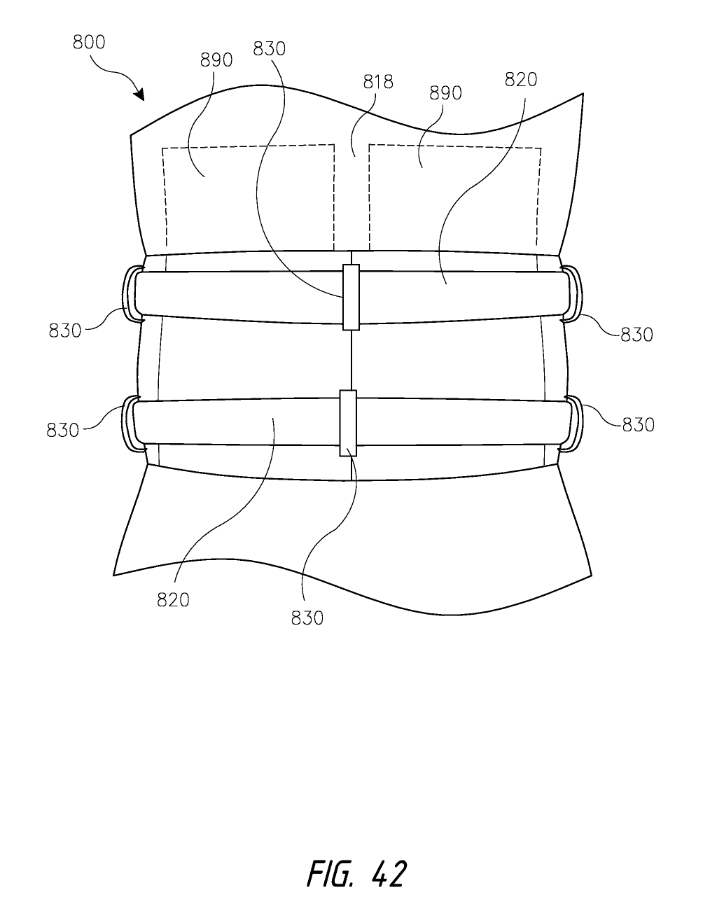

FIGS. 40-42 illustrate a lumbar/sacral therapy garment 800, according to embodiments of the present invention. Garment 800 in these depictions includes a tubular sleeve 810, a slit 818, straps 820, guide loop attachments 830, ladder lock buckles 840, and flaps 890, according to embodiments of the present invention. The tubular sleeve 810 may be slid over the torso of the user. In some embodiments, the tubular sleeve 810 begins as a flat wrap which is wrapped around the torso of the user and connected to itself. The guide loop attachments 830 provide placement for the straps 820 secured around the user's torso. The varied placement of guide loop attachments 830 permits a custom number of straps 820 to be used, as well as custom placement of those straps 820 according to numerous possible combinations and placements as desired to enhance therapy or attachment strength, according to embodiments of the present invention. As described above, the tubular sleeve 810 may be or have integrated therewith a thermal pack, such as a gel pack, for hot or cold therapy. Alternatively, tubular sleeve 810 may include a pocket to receive a thermal pack; the pocket may open at flap 890 which may extend beyond the pocket like the top flap of an envelope. In some embodiments, the flap 890 extends from the side of the pocket that goes against a user's skin, so that the flap 890, when folded back into the pocket and/or under the strap 820 does not form a double insulating layer between the thermal pack and the user's skin, according to embodiments of the present invention. In some embodiments, the flap 890 may include a slit 818 for ease of use.

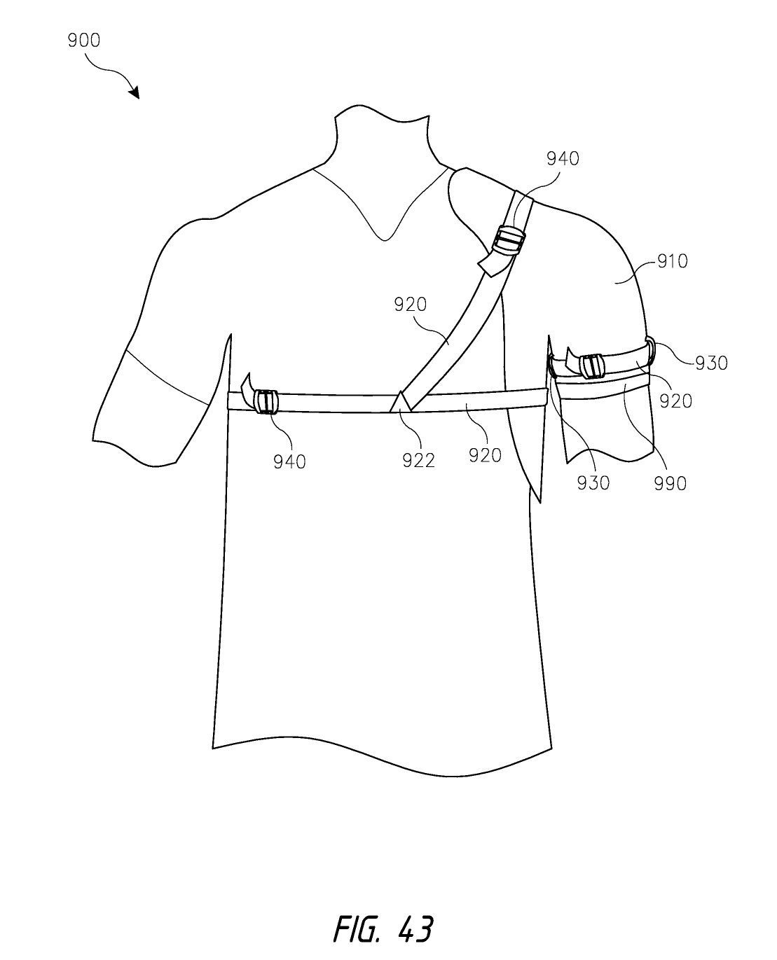

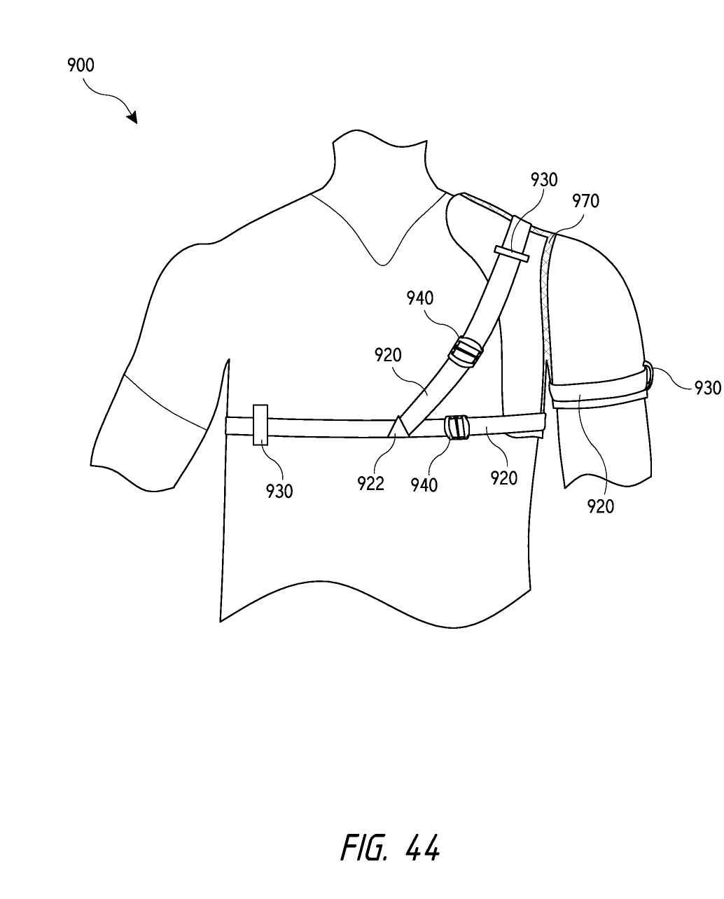

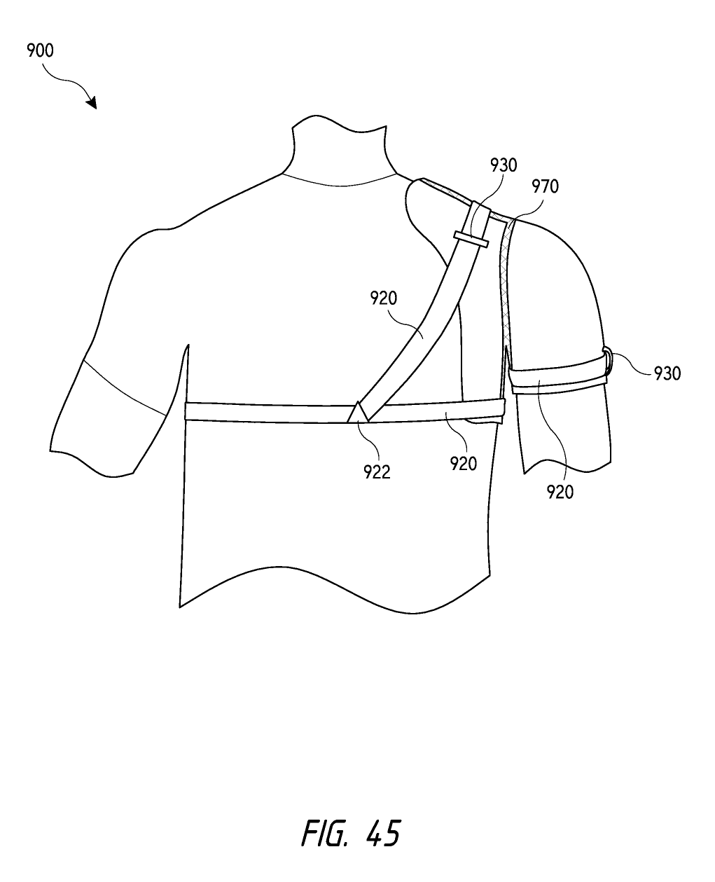

FIGS. 43-45 illustrate a shoulder therapy garment 900, according to embodiments of the present invention. Garment 900 in these depictions includes a tubular sleeve 910, straps 920, guide loop attachments 930, ladder lock buckles 940, and elastic inserts 970, according to embodiments of the present invention. The tubular sleeve 910 may be slid over the arm and shoulder of the user. The guide loop attachments 930 provide placement for the straps 920 secured around the user's torso and/or upper arm. The varied placement of guide loop attachments 930 permits a custom number of straps 920 to be used, as well as custom placement of those straps 920 according to numerous possible combinations and placements as desired to enhance therapy or attachment strength, according to embodiments of the present invention. As described above, the tubular sleeve 910 may be or have integrated therewith a thermal pack, such as a gel pack, for hot or cold therapy. Alternatively, tubular sleeve 910 may include a pocket to receive a thermal pack. The particular strap system depicted in FIGS. 43-45 permits straps 920 around the torso and upper arm to be adjusted independently for tightness and fit, while permitting the strap 920 that extends from the torso strap and up around the user's shoulder to be adjusted for the height or placement of the garment 900, according to embodiments of the present invention. The straps 920 extending over the shoulder and around the torso may be connected to each other via a triglide device 922 which holds the straps together and permits the shoulder strap 920 position to be adjusted with respect to the torso strap 920 position. Such a triglide device 922 may be used to couple the shoulder strap to the torso strap on one or both of the anterior and posterior sides of the garment 900, according to embodiments of the present invention.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A therapeutic digit cover, comprising:
a tubular sleeve for one or more digits, wherein the tubular sleeve has an opening and a length and is closed along at least part of the length;
a thermal pack incorporated in the tubular sleeve, the thermal pack is fully enclosed in the tubular sleeve such that there is no direct contact of the thermal pack with a user's skin when the therapeutic digit cover is worn by the user;
a strap with a closed loop, wherein the closed loop is configured to encircle a palm of the hand between an index finger and a thumb of the user when the therapeutic digit cover is worn by the user;
at least one guide loop attachment to hold the strap in place on the user's hand.

2. The therapeutic digit cover of claim 1, wherein the tubular sleeve is conical.

3. The therapeutic digit cover of claim 1, wherein the tubular sleeve has a pocket configured to accept the thermal pack.

4. The therapeutic digit cover of claim 3, wherein the pocket is an open pocket.

5. The therapeutic digit cover of claim 3, wherein the pocket is a stitched closed pocket.

6. The therapeutic digit cover of claim 3, wherein the pocket is a different color than a color of the therapeutic digit cover.

7. The therapeutic digit cover of claim 1, wherein the thermal pack comprises one or more of: gel, flax seed, rice, and corn.

8. The therapeutic digit cover of claim 1, wherein the opening is a first opening, and wherein the tubular sleeve has a second opening for a second digit of the hand of the user.

9. The therapeutic digit cover of claim 1, wherein the tubular sleeve is configured to accept at least one finger of the hand of the user.

10. The therapeutic digit cover of claim 1, wherein the tubular sleeve comprises elastic inserts.

11. The therapeutic digit cover of claim 1, wherein the guide loop attachment is a patch that allows for compression to be administered in specific areas of the user's hand.

12. The therapeutic digit cover of claim 1 wherein the guide loop attachment has a width from 0.1 inches to 2 inches.

13. The therapeutic digit cover of claim 1, wherein the strap is adjustable.

14. The therapeutic digit cover of claim 1, wherein the strap includes a ladder lock buckle for adjustment of the strap.

15. The therapeutic digit cover of claim 1, wherein the tubular sleeve extends up to a distal phalanx of the digit.

16. A method of administering therapy to a digit of a user, the method comprising:
positioning a therapeutic digit cover comprising a tubular sleeve to cover at least one digit, wherein the tubular sleeve has an opening and is closed along at least part of its length;
chilling or heating a thermal pack;
securing the therapeutic digit cover with a strap, wherein the strap is a closed loop that encircles a palm of a hand between an index finger and a thumb of the user;
adjusting the strap to apply pressure to a desired portion of the therapeutic digit cover.

17. The method of administering therapy of claim 16, wherein adjusting the strap includes tightening and loosening the strap using a ladder lock buckle fastener.

18. The method of administering therapy to a digit of claim 16, wherein adjusting the strap includes looping the strap through one or more guide loop attachments to hold the strap in place.

19. The method of administering therapy to a digit of claim 16, wherein the therapeutic digit cover is a first therapeutic digit cover, the method further comprising positioning a second therapeutic digit cover on the hand of the user.

20. A therapeutic digit cover kit comprising:
a first digit cover having a distal end and a proximal end, the proximal end comprising a first guide loop attachment;
a second digit cover having a distal end and a proximal end, the proximal end comprising a second guide loop attachment, wherein the first digit cover is independent of the second digit cover;
a strap configured to form a closed loop, wherein the closed loop is configured to encircle the palm of a hand of a user between an index finger and thumb of the user;
wherein the strap is selectively insertable through one or both of the first guide loop and second guide loop attachment for customizing a number of digits covered.

21. The therapeutic digit cover kit of claim 20, wherein the strap comprises a ladder lock buckle fastener.

22. The therapeutic digit cover kit of claim 20, further comprising a third digit cover having a distal end and a proximal end, the proximal end comprising a third guide loop attachment, wherein the third digit cover is independent of the first and second digit covers, and wherein the strap is selectively insertable through one or more of the first, second, and third guide loop attachments for customizing the number of digits covered.

23. The therapeutic digit cover kit of claim 20, further comprising a fourth digit cover having a distal end and a proximal end, the proximal end comprising a fourth guide loop attachment, wherein the fourth digit cover is independent of the first, second, and third digit covers, and wherein the strap is selectively insertable through one or more of the first, second, third, and fourth guide loop attachments for customizing the number of digits covered.
